(19) United States
(12) Patent Application Publication
(10) Pub. No.: US 2005/0123545 A1
(43) Pub. Date: Jun. 9, 2005

(54) Diagnosis and treatment of malignant neoplasms

(76) Inventors: Jack R. Wands, Waban, MA (US); Suzanne M. de la Monte, East Greenwich, RI (US); Alan H. Deutch, Columbia, MD (US); Hossein A. Ghanbari, Potomac, MD (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/918,685

(22) Filed: Aug. 13, 2004

Related U.S. Application Data
(63) Continuation-in-part of application No. 09/436,184, filed on Nov. 8, 1999.
(60) Provisional application No. 60/494,896, filed on Aug. 13, 2003.

(51) Int. Cl. A61K 39/395; C12P 21/04; C12N 9/64

(52) U.S. Cl. 424/146.1; 435/70.21; 435/226

(57) ABSTRACT

The invention features a method of inhibiting tumor growth and/or tumor invasiveness in a mammal by administering to a mammal a compound (e.g., an antagonistic antibody) which inhibits expression or enzymatic activity of human aspartyl (asparaginyl) beta-hydroxylase (HAAH). The invention also features a method for diagnosing the growth of a malignant neoplasm (e.g., pancreatic cancer) in a mammal by contacting a tissue or bodily fluid from the mammal with an antibody which binds to a HAAH polypeptide under conditions sufficient to form an antigen-antibody complex and/or detecting the antigen-antibody complex.

### RELATED APPLICATIONS

This application claims priority to provisional application U.S. Ser. No. 60/494,896, filed Aug. 13, 2003, which is herein incorporated by reference in its entirety.

### BACKGROUND OF THE INVENTION

Cancer currently constitutes the second most common cause of death in the United States. Carcinomas of the pancreas are the eighth most prevalent form of cancer and fourth among the most common causes of cancer deaths in this country. The incidence of pancreatic cancer has been increasing steadily in the past twenty years in most industrialized countries, exhibiting the characteristics of a growing epidemiological problem.

The prognosis for pancreatic carcinoma is, at present, very poor, it displays the lowest five-year survival rate among all cancers. Such prognosis results primarily from delayed diagnosis, due in part to the fact that the early symptoms are shared with other more common abdominal ailments. The diagnosis of pancreatic cancer is often dependent on exploratory surgery, inevitably performed after the disease has advanced considerably.

Substantial efforts have been directed to developing tools useful for early diagnosis of pancreatic carcinomas. Nonetheless, a definitive diagnosis is often dependent on exploratory surgery which is inevitably performed after the disease has advanced past the point when early treatment may be effected. One promising method for early diagnosis of various forms of cancer is the identification of specific biochemical moieties, termed antigens present on the surface of cancerous cells. Antibodies which will specifically recognize and bind to the antigens present on the surfaces of cancer cells potentially provide powerful tools for the diagnosis and treatment of the particular malignancy. Tumor specific cell surface antigens have previously been identified for certain melanomas, lymphomas malignancies of the colon and reproductive tract.

There thus exists a great and long-felt need for a cell surface marker which is present on the surface of neoplastic cells of the pancreas, and for antibodies which specifically recognize such a cell surface marker. Such markers and corresponding antibodies would be useful not only in the early detection of pancreatic cancers, but in their treatment as well. The present invention satisfies these needs and provides related advantages as well.

Human aspartyl (asparaginyl) beta-hydroxylase (HAAH) is normally localized to the endoplasmic reticulum, however upon cellular transformation it is translocated to the cell surface. Over-expression of the enzyme human aspartyl (asparaginyl) β-hydroxylase (HAAH) has been detected in many cancers tested including lung, liver, colon, pancreas, prostate, ovary, bile duct, and breast. HAAH is highly specific for cancer and is not significantly present in adjacent non-affected tissue, or in tissue samples from normal individuals. HAAH functions to hydroxylate aspartyl or asparaginyl residues within EGF-like domains of specific proteins. While the natural substrates of HAAH remain unknown, potential target proteins containing EGF-like domains include those involved in cellular signaling (e.g., notch) and/or cell/extra-cellular matrix interactions (e.g., tenascin).

It has previously been shown that over-expression of HAAH is sufficient to induce cellular transformation, increase cellular motility and invasiveness, and establish tumor formation in vivo; even a partial inhibition of HAAH expression can reverse these effects. Thus, the over-expression of HAAH in tumor cells and its functional relevance to tumorigenesis, growth and metastasis represent an important and novel target for cancer therapy. Recently, the inhibition of tumor cell migration utilizing HAAH-directed antisense oligonucleotides was reported. The cell surface localization of HAAH in cancerous tissue lends to the application of an antibody-based approach.

### SUMMARY OF THE INVENTION

A preferred embodiment of the instant invention is a method of inhibiting tumor growth and/or tumor invasiveness in a mammal, which is carried out by administering to the mammal a compound (e.g., an antibody) which inhibits expression (intra- or extracellular) of HAAH and/or inhibits enzymatic activity (e.g., hydroxylation of an epidermal growth factor (EGF)-like domain of a polypeptide) of HAAH and/or causes the destruction (e.g., cell-mediated destruction (such as T-cell mediated killing)) of cells expressing HAAH (preferably those expressing HAAH on the surface).

As described in Example 8, HAAH has been shown to be differentially expressed on the surface of pancreatic cancer cells versus normal pancreatic cells. Therefore, one preferred embodiment of the invention is a method of treating pancreatic cancer by targeting the destruction of pancreatic cancer cells expressing HAAH on the surface of the cell. In addition, a preferred antibody of the present invention specifically or preferentially binds HAAH on the surface of pancreatic cancer cells and inhibits expression (intra- or extracellular) of HAAH and/or inhibits enzymatic activity (e.g., hydroxylation of an epidermal growth factor (EGF)-like domain of a polypeptide) of HAAH and/or causes the destruction (e.g., cell-mediated destruction (such as T-cell mediated killing)) of cells expressing HAAH. Another preferred embodiment is a method for diagnosing pancreatic cancer in a mammal by contacting pancreatic tissue or bodily fluid from the mammal with an antibody which binds to a HAAH polypeptide under conditions sufficient to form an antigen-antibody complex and/or detecting the antigen-antibody complex.

The invention also features a method for diagnosing the growth of other malignant neoplasms in a mammal by contacting a tissue or bodily fluid from the mammal with an antibody which binds to a HAAH polypeptide under conditions sufficient to form an antigen-antibody complex and/or detecting the antigen-antibody complex. Malignant neoplasms detected in this manner include, for example, liver cancer, colon cancer, breast cancer, and cancer of the bile ducts. Neoplasms of the central nervous system (CNS) such as primary malignant CNS neoplasms of both neuronal and glial cell origin and metastatic CNS neoplasms are also detected. Brain cancers include metastatic brain tumors, as well as primary brain tumors such as glioma, astrocytomas, and hemangiomas. Patient derived tissue samples, e.g., biopsies of solid tumors, as well as bodily fluids such as a CNS-derived bodily fluid, blood, serum, urine, saliva, sputum, lung effusion, and ascites fluid, are contacted with an HAAH-specific antibody.

The invention further features a method for preventing or inhibiting the growth of a malignant neoplasm in a mammal by contacting a tissue or bodily fluid from the mammal with an antibody which binds to an human aspartyl (asparaginyl) beta-hydroxylase (HAAH).

The invention includes a method of eliciting an immune response or conferring an immune response to a tumor cell, e.g., a pancreatic tumor, in a mammal by administering to a mammal an antibody which binds to HAAH or a polynucleotide encoding such an antibody.

Preferably, an antibody of the invention binds to a site in an extracellular domain (e.g., a site within residues 1-700) of HAAH. Antibodies of the invention may also bind to an ectodomain of HAAH (residues 19-75 of SEQ ID NO:2). More preferably an antibody of the invention binds to a catalytic domain of HAAH, e.g., amino acids 650-700 of SEQ ID NO:2. For example, FB50 binds to a polypeptide with the amino acid sequence NPVEDS (residues 286-291 of SEQ ID NO:2). Monoclonal antibody HBOH1 binds to a polypeptide with the amino acid sequence QPWWTPK (residues 573-579 of SEQ ID NO:2), and monoclonal antibody HBOH-2 binds to a polypeptide containing the amino acid sequence LPEDENLR (residues 613-620 of SEQ ID NO:2). The foregoing antigenic epitopes of HAAH are located on the cell surface of malignant cells. Other HAAH-specific antibodies suitable for passive immunization include 5C7, 5E9, 19B, 48A, 74A, 78A, 86A, HA238A, HA221, HA 239, HA241, HA329, and HA355.

In another embodiment, the antibody binds to the extracellular domain of HAAH, preferably with a K_{off} of less than 1×10⁻³ s⁻¹, more preferably less than 3×10⁻³ s⁻¹. In other embodiments, the antibody binds to EphA4 with a K_{off} of less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ S-1, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

Antibodies of the invention include, but are not limited to, monoclonal antibodies, synthetic antibodies, recombinantly produced antibodies, intrabodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies used in the methods of the present invention include immunoglobulin molecules and immunologically active portions of immunoglobulin molecules, i.e., molecules that contain an antigen binding site that immunospecifically binds to HAAH and inhibits or reduces a cancer cell phenotype, preferentially binds an HAAH epitope exposed on cancer cells but not non-cancer cells, and/or binds HAAH with a K_{off} of less than 3×10⁻³ s⁻¹. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animals that express antibodies from human genes.

The antibodies used in the methods of the present invention may be monospecific, bispecific, trispecific or of greater multispecificity. Multispecific antibodies may immunospecifically bind to different epitopes of an HAAH polypeptide or may immunospecifically bind to both an HAAH polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

In a preferred embodiment, antibodies of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In an embodiment of this invention, an antibody or ligand that immunospecifically binds to HAAH will comprise a portion of the BiTE molecule. For example, the VH and/or VL (preferably a scFV) of an antibody that binds HAAH can be fused to an anti-CD3 binding portion such as that of the molecule described above, thus creating a BiTE molecule that targets HAAH. In addition to the variable heavy and or light chain of antibody against HAAH, other molecules that bind HAAH can comprise the BiTE molecule. In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3). In one embodiment of the invention, the BiTE molecule targets the HAAH-expressing cancer cell for destruction (e.g., T cell-mediated killing). In one embodiment of the invention, the BiTE molecule is a HAAH agonist. In another embodiment, the BiTE molecule is a HAAH antagonist.

The antibody of the invention to be administered is a heterodimeric antibody, a single chain antibody, or a high affinity single chain antibody. By high affinity is meant that the antigen-specific binding affinity of the antibody has a K_{d} in the nanomolar range. Preferably, the binding affinity is in the range of 100 pM or higher affinity. For example, the antibody, antibody fragment, or single chain antibody has an antigen-specific binding affinity in the range of 10⁻¹⁰ to 10⁻¹⁵ molar.

The antibody, or fragment thereof, activates complement in a patient treated with the antibody. Preferably, the antibody mediates antibody-dependent cytotoxicity of tumor cells in the patient treated with the antibody. The antibody, or fragment thereof, is administered alone or conjugated to a cytotoxic agent. In the latter case, binding of the antibody to a tumor cell results in impairment or death of the cell, thereby reducing tumor load. The antibody is conjugated to a radiochemical, or a chemical tag which sensitizes the cell to which it is bound to radiation or laser-mediated killing.

Also within the scope of the invention are methods of inducing an HAAH-specific immune response to reduce tumor growth by active immunization. The method involves administering to a mammal an HAAH polypeptide, e.g., a polypeptide containing the amino acid sequence of SEQ ID NO:2. Immunogenic HAAH fragments are also administered to generate an immune response to a particular portion of HAAH. For example, to generate an antibody response to HAAH on the surface of cells, a polypeptide containing an extracellular domain of HAAH (but lacking an intracellular domain of HAAH) is administered. To generate antibodies, which inhibit HAAH activity, the individual is immunized with a polypeptide containing a catalytic domain of HAAH (e.g., amino acids 650-700 of SEQ ID NO:2). Optionally, the polypeptide compositions contain a clinically-acceptable adjuvant compound. Such adjuvants are generally known in the art, and include oil-emulsions, Freunds Complete and Incomplete adjuvant, Vitamin E, aluminum salts or gels, such as aluminum hydroxide, -oxide or -phosphate, saponins, polymers based on polyacrylic acid, such as carbopols, non-ionic block polymers, fatty acid amines, such as pyridin and DDA, polymers based on dextran, such as dextran sulphate and DEAE dextran, muramyldipeptides, ISCOMs (immune stimulating complexes, e.g., as described in European Patent EP 109942), biodegradable microcapsules, liposomes, bacterial immune stimulators, such as MDP and LPS, and glucans. Other adjuvant compounds are known in the art, e.g., described in Altman and Dixon, 1989, Advances in Veterinary Science and Comparative Medicine 33: 301-343). Alum is preferred for human use.

An HAAH-specific immune response is also induced by administering to a mammal a polynucleotide composition encoding an HAAH polypeptide, or a degenerate variant of the HAAH-encoding polynucleotide. For example, the polynucleotide contains the nucleotide sequence of SEQ ID NO:3, or a degenerate variant thereof, or a fragment thereof encoding a specific immunogenic domain of HAAH. Preferably, the HAAH polypeptide encoded by the polynucleotide (or directly administered polypeptide) is enzymatically nonfunctional. More preferably, the HAAH polynucleotide encodes an HAAH polypeptide that is secreted, e.g., the construct contains a signal sequence for transport out of the cell and into an extracellular space. The HAAH polypeptide lacks an essential histidine. The HAAH polypeptide is a truncated HAAH, which contains the first 650 amino acids of SEQ ID NO:2.

Optionally, the polynucleotide composition contains a transfection-enhancing agent, such as a precipitating agent or a lipid. Preferably, the encoded HAAH polypeptide contains the amino acid sequence of SEQ ID NO:2 (full-length HAAH) or a fragment thereof, which contains an extracellular domain of HAAH and lacks an intracellular domain of HAAH. Preferably, the polynucleotide contains a catalytic domain of HAAH. The HAAH-encoding sequences are operably-linked to a promoter and other regulatory sequences for expression of the polypeptides in target cells. The polypeptide may be directed intracellularly or marked for extracellular expression, or secretion. The polynucleotide directs expression in a target cell, which expresses appropriate accessory molecules for antigen presentation, e.g., major histocompatibility antigens.

Methods for diagnosis include detecting a tumor cell in bodily fluids as well as detecting a tumor cell in tissue (in vivo or ex vivo). For example, a biopsied tissue is contacted with an HAAH-specific antibody and antibody binding measured. Whole body diagnostic imaging may be carried out to detect microtumors undetectable using conventional diagnostic methods. Accordingly, a method for diagnosing a neoplasm in a mammal is carried out by contacting a tissue, e.g., a lymph node, of a mammal with a detectably-labeled antibody which binds to HAAH. An increase in the level of antibody binding at a tissue site compared to the level of binding to a normal normeoplastic tissue indicates the presence of a neoplasm at the tissue site. For detection purposes, the antibody (or HAAH-binding fragment thereof) is labeled with a non-radioactive tag, a radioactive compound, or a colorimetric agent. For example, the antibody or antibody fragment is tagged with 125I, ⁹⁹Tc, Gd⁺⁺⁺, or Fe⁺⁺. Green fluorescent protein is used as a colorimetric tag.

The invention also includes a soluble fragment of HAAH. The soluble HAAH polypeptide contains an extracellular domain and optionally lacks part or all of the cytoplasmic domain or transmembrane domain of HAAH. In one example, the fragment lacks residues 660-758 of SEQ ID NO:2. In another example, the fragment lacks residues 679-697 (His motif) of SEQ ID NO:2. In yet another example, the fragment, lacks at least one residue of SEQ ID NO:2, the residue being selected from the group consisting of residue 661, 662, 663, 670, 671, 672, and 673. An HAAH fragment is an HAAH polypeptide, the length of which is less than that of a full-length HAAH protein. The full-length HAAH protein is shown in Table 1.

Diagnostic kits are also encompassed by the invention. For example, a kit for detecting a tumor cell contains an antibody, or fragment thereof, which binds to HAAH. The kit optionally contains a means for detecting binding of the antibody to the tumor cell. For example, the kit contains a detectable marker, e.g., a nonradioactive marker such as Gd⁺⁺⁺ or Fe⁺⁺ or a radioactive compound. The kit may also contain instructions for use, a standard reagent for determining positive antibody binding, or a negative control for determining lack of antibody binding. The components are packaged together in a kit.

The assay format described herein is useful to generate temporal data used for prognosis of malignant disease. A method for prognosis of a malignant neoplasm of a mammal is carried out by (a) contacting a bodily fluid from the mammal with an antibody which binds to an HAAH polypeptide under conditions sufficient to form an antigen-antibody complex and detecting the antigen-antibody complex; (b) quantitating the amount of complex to determine the level of HAAH in the fluid; and (c) comparing the level of HAAH in the fluid with a normal control level of HAAH. An increasing level of HAAH over time indicates a progressive worsening of the disease, and therefore, an adverse prognosis.

The invention also includes an antibody which binds to HAAH. The antibody preferably binds to a site in the carboxyterminal catalytic domain of HAAH. Alternatively, the antibody binds to an epitope that is exposed on the surface of the cell. The antibody is a polyclonal antisera or monoclonal antibody. The invention encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin. Preferably the antibody is a monoclonal antibody such as FB50, 5C7, 5E9, 19B, 48A, 74A, 78A, 86A, HA238A, HA221, HA 239, HA241, HA329, or HA355. Antibodies which bind to the same epitopes as those monoclonal antibodies are also within the invention.

A HAAH-specific intrabody is a recombinant single chain HAAH-specific antibody that is expressed inside a target cell, e.g., tumor cell. Such an intrabody binds to endogenous intracellular HAAH and inhibits HAAH enzymatic activity or prevents HAAH from binding to an intracellular ligand. HAAH-specific intrabodies inhibit intracellular signal transduction, and as a result, inhibit growth of tumors which overexpress HAAH.

A kit for diagnosis of a tumor in a mammal contains an HAAH-specific antibody. The diagnostic assay kit is preferentially formulated in a standard two-antibody binding format in which one HAAH-specific antibody captures HAAH in a patient sample and another HAAH-specific antibody is used to detect captured HAAH. For example, the capture antibody is immobilized on a solid phase, e.g., an assay plate, an assay well, a nitrocellulose membrane, a bead, a dipstick, or a component of an elution column. The second antibody, i.e., the detection antibody, is typically tagged with a detectable label such as a colorimetric agent or radioisotope.

Also within the scope of the invention is a method of inhibiting tumor growth and/or tumor invasiveness in a mammal, which is carried out by administering to the mammal a compound (e.g., an antagonistic antibody) which inhibits expression or enzymatic activity of HAAH.

Most preferably, the compound is an antibody or portion thereof which preferentially or specifically binds HAAH on the surface of tumor cells. See, Passive Immunization infra.

In an alternative embodiment, the compound is a substantially pure nucleic acid molecule such as an HAAH antisense DNA, the sequence of which is complementary to a coding sequence of HAAH. Expression of HAAH is inhibited by contacting mammalian cells, e.g., tumor cells, with HAAH antisense DNA or RNA, e.g., a synthetic HAAH antisense oligonucleotide. The sequence of the antisense is complementary to a coding or noncoding region of a HAAH gene. For example, the sequence is complementary to a nucleotide sequence in the 5′ untranslated region of a HAAH gene. Examples of HAAH antisense oligonucleotides which inhibit HAAH expression in mammalian cells include oligonucleotides containing SEQ ID NO:10, 11, or 12. An HAAH antisense nucleic acid is introduced into glioblastoma cells or other tumor cells which overexpress HAAH. Binding of the antisense nucleic acid to an HAAH transcript in the target cell results in a reduction in HAAH production by the cell. By the term “antisense nucleic acid” is meant a nucleic acid (RNA or DNA) which is complementary to a portion of an mRNA, and which hybridizes to and prevents translation of the mRNA. Preferably, the antisense DNA is complementary to the 5′ regulatory sequence or the 5′ portion of the coding sequence of HAAH mRNA (e.g., a sequence encoding a signal peptide or a sequence within exon 1 of the HAAH gene). Standard techniques of introducing antisense DNA into the cell may be used, including those in which antisense DNA is a template from which an antisense RNA is transcribed. The method is to treat tumors in which expression of HAAH is upregulated, e.g., as a result of malignant transformation of the cells. The length of the oligonucleotide is at least 10 nucleotides and may be as long as the naturally-occurring HAAH transcript. Preferably, the length is between 10 and 50 nucleotides, inclusive. More preferably, the length is between 10 and 20 nucleotides, inclusive.

By “substantially pure DNA or RNA” is meant that the nucleic acid is free of the genes which, in the naturally-occurring genome of the organism from which the DNA of the invention is derived, flank a HAAH gene. The term therefore includes, for example, a recombinant nucleic acid which is incorporated into a vector, into an autonomously replicating plasmid or virus, or into the genomic DNA of a procaryote or eucaryote at a site other than its natural site; or which exists as a separate molecule (e.g., a cDNA or a genomic or cDNA fragment produced by PCR or restriction endonuclease digestion) independent of other sequences. It also includes a recombinant nucleic acid which is part of a hybrid gene encoding additional polypeptide sequence such as a nucleic acid encoding an chimeric polypeptide, e.g., one encoding an antibody fragment linked to a cytotoxic polypeptide. Alternatively, HAAH expression is inhibited by administering a ribozyme or a compound which inhibits binding of Fos or Jun to an HAAH promoter sequence.

Compounds, which inhibit an enzymatic activity of HAAH, are useful to inhibit tumor growth in a mammal. By enzymatic activity of HAAH is meant hydroxylation of an epidermal growth factor (EGF)-like domain of a polypeptide. For example an EGF-like domain has the consensus sequence CX₇CX₄CX₁₀CXCX₈C (SEQ ID NO:1). HAAH hydroxylase activity is inhibited intracellularly. For example, a dominant negative mutant of HAAH (or a nucleic acid encoding such a mutant) is administered. The dominant negative HAAH mutant contains a mutation which changes a ferrous iron binding site from histidine of a naturally-occurring HAAH sequence to a non-iron-binding amino acid, thereby abolishing the hydroxylase activity of HAAH. The histidine to be mutated, e.g., deleted or substituted, is located in the carboxyterminal catalytic domain of HAAH. For example, the mutation is located between amino acids 650-700 (such as the His motif, underlined sequence of SEQ ID NO:2) the native HAAH sequence. For example, the mutation is at residues 671, 675, 679, or 690 of SEQ ID NO:2. An HAAH-specific intrabody is also useful to bind to HAAH and inhibit intracellular HAAH enzymatic activity, e.g., by binding to an epitope in the catalytic domain of HAAH. Other compounds such as L-mimosine or hydroxypyridone are administered directly into a tumor site or systemically to inhibit HAAH hydroxylase activity.

TABLE 1
Amino acid sequence of HAAH

| | |
|---|---|
| MAQRKNAKSS GNSSSSGSGS GSTSAGSSSP GARRETKHGG HKNGRKGGLS GTSFFTWFMV | 61 |
| IALLGVWTSV AVVWFDLVDY EEVLGKLGIY DADGDGDFDV DDAKVLLGLK ERSTSEPAVP | 121 |
| PEEAEPHTEP EEQVPVEAEP QNIEDEAKEQ IQSLLHEMVH AEHVEGEDLQ QEDGPTGEPQ | 181 |
| QEDDEFLMAT DVDDRFETLE PEVSHEETEH SYHVEETVSQ DCNQDMEEMM SEQENPDSSE | 241 |
| PVVEDERLHH DTDDVTYQVY EEQAVYEPLE NEGIEITEVT APPEDNPVED SQVIVEEVSI | 301 |
| FPVEEQQEVP PETNRKTDDP EQKAKVKKKK PKLLNKFDKT IKAELDAAEK LRKRGKIEEA | 361 |
| VNAFKELVRK YPQSPRARYG KAQCEDDLAE KRRSNEVLRG AIETYQEVAS LPDVPADLLK | 421 |
| LSLKRRSDRQ QFLGHMRGSL LTLQRLVQLF PNDTSLKNDL GVGYLLIGDN DNAKKVYEEV | 481 |
| LSVTPNDGFA KVHYGFILKA QNKIAESIPY LKEGIESGDP GTDDGRFYFH LGDAMQRVGN | 541 |
| KEAYKWYELG HKRGHFASVW QRSLYNVNGL KAQPWWTPKE TGYTELVKSL ERNWKLIRDE | 601 |
| GLAVMDKAKG LFLPEDENLR EKGDWSQFTL WQQGRRNENA CKGAPKTCTL LEKFPETTGC | 661 |
| RRGQIKYSIM HPGTHVWPHT GPTNCRLRMH LGLVIPKEGC KIRCANETRT WEEGKVLIFD | 721 |
| DSFEHEVWQD ASSFRLIFIV DVWHPELTPQ QRRSLPAI | |

(SEQ ID NO: 2; GENBANK Accession No. S83325; His motif is underlined; conserved sequences within the catalytic domain are designated by bold type)

For example, a compound which inhibits HAAH hydroxylation is a polypeptide that binds a HAAH ligand but does not transduce an intracellular signal or an polypeptide which contains a mutation in the catalytic site of HAAH. Such a polypeptide contains an amino acid sequence that is at least 50% identical to a naturally-occurring HAAH amino acid sequence or a fragment thereof and which has the ability to inhibit HAAH hydroxylation of substrates containing an EGF-like repeat sequence. More preferably, the polypeptide contains an amino acid sequence that is at least 75%, more preferably at least 85%, more preferably at least 95% identical to SEQ ID NO:2.

A substantially pure HAAH polypeptide or HAAH-derived polypeptide such as a mutated HAAH polypeptide is preferably obtained by expression of a recombinant nucleic acid encoding the polypeptide or by chemically synthesizing the protein. A polypeptide or protein is substantially pure when it is separated from those contaminants which accompany it in its natural state (proteins and other naturally-occurring organic molecules). Typically, the polypeptide is substantially pure when it constitutes at least 60%, by weight, of the protein in the preparation. Preferably, the protein in the preparation is at least 75%, more preferably at least 90%, and most preferably at least 99%, by weight, HAAH. Purity is measured by any appropriate method, e.g., column chromatography, polyacrylamide gel electrophoresis, or HPLC analysis. Accordingly, substantially pure polypeptides include recombinant polypeptides derived from a eucaryote but produced in E. coli or another procaryote, or in a eucaryote other than that from which the polypeptide was originally derived.

Nucleic acid molecules which encode such HAAH or HAAH-derived polypeptides are also within the invention.

TABLE 2
HAAH cDNA sequence

| | |
|---|---|
| cggaccgtgc aatggcccag cgtaagaatg ccaagagcag cggcaacagc agcagcagcg | 61 |
| gctccggcag cggtagcacg agtgcgggca gcagcagccc cggggcccgg agagagacaa | 121 |
| agcatggagg acacaagaat gggaggaaag gcggactctc gggaacttca ttcttcacgt | 181 |
| ggtttatggt gattgcattg ctgggcgtct ggacatctgt agctgtcgtt tggtttgatc | 241 |
| ttgttgacta tgaggaagtt ctaggaaaac taggaatcta tgatgctgat ggtgatggag | 301 |
| attttgatgt ggatgatgcc aaagttttat taggacttaa agagagatct acttcagagc | 361 |
| cagcagtccc gccagaagag gctgagccac acactgagcc cgaggagcag gttcctgtgg | 421 |
| aggcagaacc ccagaatatc gaagatgaag caaaagaaca aattcagtcc cttctccatg | 481 |
| aaatggtaca cgcagaacat gttgagggag aagacttgca acaagaagat ggacccacag | 541 |
| gagaaccaca acaagaggat gatgagtttc ttatggcgac tgatgtagat gatagatttg | 601 |
| agaccctgga acctgaagta tctcatgaag aaaccgagca tagttaccac gtggaagaga | 661 |
| cagtttcaca agactgtaat caggatatgg aagagatgat gtctgagcag gaaaatccag | 721 |
| attccagtga accagtagta gaagatgaaa gattgcacca tgatacagat gatgtaacat | 781 |
| accaagtcta tgaggaacaa gcagtatatg aacctctaga aaatgaaggg atagaaatca | 841 |
| cagaagtaac tgctccccct gaggataatc ctgtagaaga ttcacaggta attgtagaag | 901 |
| aagtaagcat ttttcctgtg gaagaacagc aggaagtacc accagaaaca aatagaaaaa | 961 |
| cagatgatcc agaacaaaaa gcaaaagtta agaaaaagaa gcctaaactt ttaaataaat | 1021 |
| ttgataagac tattaaagct gaacttgatg ctgcagaaaa actccgtaaa aggggaaaaa | 1081 |
| ttgaggaagc agtgaatgca tttaaagaac tagtacgcaa ataccctcag agtccacgag | 1141 |
| caagatatgg gaaggcgcag tgtgaggatg atttggctga gaagaggaga agtaatgagg | 1201 |
| tgctacgtgg agccatcgag acctaccaag aggtggccag cctacctgat gtccctgcag | 1261 |
| acctgctgaa gctgagtttg aagcgtcgct cagacaggca acaatttcta ggtcatatga | 1321 |
| gaggttccct gcttaccctg cagagattag ttcaactatt tcccaatgat acttccttaa | 1381 |
| aaaatgacct tggcgtggga tacCtcttga taggagataa tgacaatgca aagaaagttt | 1441 |
| atgaagaggt gctgagtgtg acacctaatg atggctttgc taaagtccat tatggcttca | 1501 |
| tcctgaaggc acagaacaaa attgctgaga gcatcccata tttaaaggaa ggaatagaat | 1561 |
| ccggagatcc tggcactgat gatgggagat tttatttcca cctgggggat gccatgcaga | 1621 |
| gggttgggaa caaagaggca tataagtggt atgagcttgg gcacaagaga ggacactttg | 1681 |
| catctgtctg gcaacgctca ctctacaatg tgaatggact gaaagcacag ccttggtgga | 1741 |
| ccccaaaaga aacgggctac acagagttag taaagtcttt agaaagaaac tggaagttaa | 1801 |
| tccgagatga aggccttgca gtgatggata aagccaaagg tctcttcctg cctgaggatg | 1861 |
| aaaacctgag ggaaaaaggg gactggagcc agttcacgct gtggcagcaa ggaagaagaa | 1921 |
| atgaaaatgc ctgcaaagga gctcctaaaa cctgtacctt actagaaaag ttccccgaga | 1981 |
| caacaggatg cagaagagga cagatcaaat attccatcat gcaccccggg actcacgtgt | 2041 |
| ggccgcacac agggcccaca aactgcaggc tccgaatgca cctgggcttg gtgattccca | 2101 |
| aggaaggctg caagattcga tgtgccaacg agaccaggac ctgggaggaa ggcaaggtgc | 2161 |
| tcatctttga tgactccttt gagcacgagg tatggcagga tgcctcatct ttccggctga | 2221 |
| tattcatcgt ggatgtgtgg catccggaac tgacaccaca gcagagacgc agccttccag | 2281 |
| caatttagca tgaattcatg caagcttggg aaactctgga gaga | |

(SEQ ID NO: 3; GENBANK Accession No. S83325; codon encoding initiating methionine is underlined).

Methods of inhibiting tumor growth also include administering a compound which inhibits HAAH hydroxylation of a NOTCH polypeptide. For example, the compound inhibits hydroxylation of an EGF-like cysteine-rich repeat sequence in a NOTCH polypeptide, e.g., one containing the consensus sequence CDXXXCXXKXGNGXCDXXCNNAACXXDGXDC (SEQ ID NO:4). Polypeptides containing an EGF-like cysteine-rich repeat sequence are administered to block hydroxylation of endogenous NOTCH.

Growth of a tumor which overexpresses HAAH is also inhibited by administering a compound which inhibits signal transduction through the insulin receptor substrate (IRS) signal transduction pathway. Preferably the compound inhibits IRS phosphorylation. For example, the compound is a peptide or non-peptide compound which binds to and inhibits phosphorylation at residues 46, 465, 551, 612, 632, 662, 732, 941, 989, or 1012 of SEQ ID NO:5. Compounds include polypeptides such those which block an IRS phosphorylation site such as a Glu/Tyr site. Antibodies such as those which bind to a carboxyterminal domain of IRS containing a phosphorylation site block IRS phosphorylation, and as a consequence, signal transduction along the pathway. Inhibition of IRS phosphorylation in turn leads to inhibition of cell proliferation. Other compounds which inhibit IRS phosphorylation include vitamin D analogue EB1089 and Wortmannin.

HAAH-overproducing tumor cells were shown to express HAAH both intracellularly and on the surface of the tumor cell. Accordingly, a method of killing a tumor cell is carried out by contacting such a tumor cell with a cytotoxic agent linked to an HAAH-specific antibody. The HAAH-specific antibody (antibody fragment, or ligand which binds to extracellular HAAH) directs the chimeric polypeptide to the surface of the tumor cell allowing the cytotoxic agent to damage or kill the tumor cell to which the antibody is bound. The monoclonal antibody binds to an epitope of HAAH such as an epitope exposed on the surface of the cell or in the catalytic site of HAAH. The cytotoxic composition preferentially kills tumor cells compared to non-tumor cell.

Screening methods to identify anti-tumor agents which inhibit the growth of tumors which overexpress HAAH are also within the invention. A screening method used to determine whether a candidate compound inhibits HAAH enzymatic activity includes the following steps: (a) providing a HAAH polypeptide, e.g., a polypeptide which contains the carboxyterminal catalytic site of HAAH; (b) providing a polypeptide comprising an EGF-like domain; (c) contacting the HAAH polypeptide or the EGF-like polypeptide with the candidate compound; and (d) determining hydroxylation of the EGF-like polypeptide of step (b). A decrease in hydroxylation in the presence of the candidate compound compared to that in the absence of the compound indicates that the compound inhibits HAAH hydroxylation of EGF-like domains in proteins such as NOTCH.

Anti-tumor agents which inhibit HAAH activation of NOTCH are identified by (a) providing a cell expressing HAAH; (b) contacting the cell with a candidate compound; and (c) measuring translocation of activated NOTCH to the nucleus of the cell. Translocation is measured by using a reagent such as an antibody which binds to a 110 kDa activation fragment of NOTCH. A decrease in translocation in the presence of the candidate compound compared to that in the absence of the compound indicates that the compound inhibits HAAH activation of NOTCH, thereby inhibiting NOTCH-mediated signal transduction and proliferation of HAAH-overexpressing tumor cells.

Nucleotide and amino acid comparisons described herein were carried out using the Lasergene software package (DNASTAR, Inc., Madison, Wis.). The MegAlign module used was the Clustal V method (Higgins et al., 1989, CABIOS 5(2):151-153). The parameter used were gap penalty 10, gap length penalty 10.

Hybridization is carried out using standard techniques, such as those described in Ausubel et al. (Current Protocols in Molecular Biology, John Wiley & Sons, 1989). “High stringency” refers to nucleic acid hybridization and wash conditions characterized by high temperature and low salt concentration, e.g., wash conditions of 65° C. at a salt concentration of 0.1×SSC. “Low” to “moderate” stringency refers to DNA hybridization and wash conditions characterized by low temperature and high salt concentration, e.g., wash conditions of less than 60° C. at a salt concentration of 1.0×SSC. For example, high stringency conditions include hybridization at 42° C. in the presence of 50% formamide; a first wash at 65° C. in the presence of 2×SSC and 1% SDS; followed by a second wash at 65° C. in the presence of 0.1%×SSC. Lower stringency conditions suitable for detecting DNA sequences having about 50% sequence identity to an HAAH gene sequence are detected by, for example, hybridization at about 42° C. in the absence of formamide; a first wash at 42° C., 6×SSC, and 1% SDS; and a second wash at 50° C., 6×SSC, and 1% SDS.

Other features and advantages of the invention will be apparent from the following description of the preferred embodiments thereof, and from the claims.

### BRIEF DESCRIPTION OF THE DRAWINGS

FIG. 1 is a bar graph showing colony formation induced by transient transfection of NIH-3T3 cells with various aspartyl (asparaginyl) beta-hydroxylase (AAH) cDNAs. Colony formation was induced by transient transfection with 10 μg DNA. In contrast, the mutant murine AAH construct without enzymatic activity has no transforming activity. The data is presented as mean number of transformed foci±SEM.

FIG. 2 is a bar graph showing the results of a densitometric analysis of a Western blot assay of proteins produced by various murine AAH stably transfected cell clones. In clones 7 and 18, there was a modest increase in HAAH gene expression, while the overexpression was to a lesser degree in clone 16.

FIGS. 3A-B are bar graphs showing colony formation in soft agar exhibited by HAAH stably transfected clones compared to HAAH enzymatic activity. FIG. 3A shows a measurement of murine AAH enzymatic activity in clones 7, 16 and 18, and FIG. 3B shows colony formation exhibited by clones 7, 16 and 18. Data is presented as mean number of colonies 10 days after plating±SEM. All three clones with modest increases in HAAH enzymatic activity, that correlated with protein expression, exhibited anchorage independent growth.

FIG. 4 is a bar graph showing tumor formation in nude mice injected with transfected clones overexpressing murine AAH. Tumor growth was assessed after 30 days. Mean tumor weight observed in mice injected with clones 7, 16 and 18 as compared to mock DNA transfected clone. All animals, which were injected with clones overexpressing HAAH, developed tumors.

FIGS. 5A-D are bar graphs showing increased AAH expression in PNET2 (FIGS. 5A, 5C) and SH-Sy5y (FIG. 5B) cells treated with retinoic acid (FIGS. 5A, 5B) or phorbol ester myristate (PMA; FIG. 5C) to induce neurite outgrowth as occurs during tumor cell invasion. The cells were treated with 10 μM retinoic acid or 100 nM PMA for 0, 1, 2, 3, 4, or 7 days. Cell lysates were analyzed by Western blot analysis using an HAAH-specific monoclonal antibody to detect the 85 kDa AAH protein. The levels of immunoreactivity were measured by volume densitometry (arbitrary units). The graphs indicate the mean±S.D. of results obtained from three separate experiments. In FIG. 5D, PNET2 cells were treated for 24 hours with sub-lethal concentrations of H₂O₂ to induce neurite retraction. Viability of greater than 90% of the cells was demonstrated by Trypan blue dye exclusion. Similar results were obtained for SH-Sy5y cells.

FIG. 6 is a bar graph showing the effects of AAH over-expression on the levels of anti-apoptosis (Bc1-2), cell cycle-mitotic inhibitor (p116 and p21/Waf1), and proliferation (proliferating cell nuclear antigen; PCNA) molecules. PNET2 neuronal cells were stably transfected with the full-length human cDNA encoding AAH (p1HAAH) or empty vector (pcDNA). AAH gene expression was under control of a CMV promoter. Western blot analysis was performed with cell lysates prepared from cultures that were 70 to 80 percent confluent. Protein loading was equivalent in each lane. Replicate blots were probed with the different antibodies. Bar graphs depict the mean S.D.'s of protein expression levels measured in three experiments. All differences are statistically significant by Student T-test analysis (P<0.01-P<0.001).

FIG. 7 is a diagram of showing the components of the IRS-1 signal transduction pathway.

FIG. 8 is a line graph showing growth curves generated in cells expressing the antisense HAAH compared to controls expressing GFP.

FIG. 9 is a diagram of the functional domains of the hIRS-1 protein and structural organization of the point mutants. All mutant and “wild type” hIRS-1 proteins construct contain a FLAG (F) epitope (DYKDDDDK; SEQ ID NO:7) at the C-terminus. PH and PTB indicate pleckstrin homology and phosphotyrosine binding, regions, respectively.

FIG. 10 is a diagram showing AAH cDNA and the location at which antisense oligonucleotides bind. The locations shown are relative to the AUG start site of the AAH cDNA.

FIG. 11 is a photograph of an electrophoretic gel showing inhibition of AAH gene expression by antisense oligonucleotide DNA molecules.

FIG. 12 is a line graph showing AAH antisense oligonucleotide binding in neuroblastoma cells.

FIG. 13 is a bar graph showing inhibition of AAH gene expression as a result of AAH antisense oligonucleotide delivery into neuroblastoma cells.

FIG. 14A is a photograph of a Western blot assay expression of NOTCH proteins.

FIG. 14B is a photograph of an electrophoretic gel showing Hes-1 gene expression as measured by reverse transcriptase-polymerase chain reaction (RT-PCR).

FIG. 14C is a photograph of a Western blot assay showing expression of NOTCH-1 and Jagged-1 under conditions in which IRS-1 signaling is reduced.

### DETAILED DESCRIPTION

HAAH is a protein belonging to the alpha-ketoglutarate dependent dioxygenase family of prolyl and lysyl hydroxylases which play a key role in collagen biosynthesis. This molecule hydroxylates aspartic acid or asparagine residues in EGF-like domains of several proteins in the presence of ferrous iron. These EGF-like domains contain conserved motifs, that form repetitive sequences in proteins such as clotting factors, extracellular matrix proteins, LDL receptor, NOTCH homologues, or NOTCH ligand homologues.

The alpha-ketoglutarate-dependent dioxygenase aspartyl (asparaginyl) beta-hydroxylase (AAH) specifically hydroxylates one aspartic or asparagine residue in EGF-like domains of various proteins. The 4.3-kb cDNA encoding the human AspH (hAspH) hybridizes with 2.6 kb and 4.3 kb transcripts in transformed cells, and the deduced amino acid sequence of the larger transcript encodes an protein of about 85 kDa. Both in vitro transcription and translation and Western blot analysis also demonstrate a 56-kDa protein that may result from posttranslational cleavage of the catalytic C-terminus.

A physiological function of AAH is the post-translational beta-hydroxylation of aspartic acid in vitamin K-dependent coagulation proteins. However, the abundant expression of AAH in several malignant neoplasms, and low levels of AAH in many normal cells indicate a role for this enzyme in malignancy. The AAH gene is also highly expressed in cytotrophoblasts, but not syncytiotrophoblasts of the placenta. Cytotrophoblasts are invasive cells that mediate placental implantation. The increased levels of AAH expression in human cholangiocarcinomas, hepatocellular carcinomas, colon cancers, and breast carcinomas were primarily associated with invasive or metastatic lesions. Moreover, overexpression of AAH does not strictly reflect increased DNA synthesis and cellular proliferation since high levels of AAH immunoreactivity were observed in 100 percent of cholangiocarcinomas, but not in human or experimental disease processes associated with regeneration or normeoplastic proliferation of bile ducts. AAH overexpression and attendant high levels of beta hydroxylase activity lead to invasive growth of transformed neoplastic cells. Detection of an increase in HAAH expression is useful for early and reliable diagnosis of the cancer types which have now been characterized as overexpressing this gene product.

Diagnosis of Malignant Tumors

HAAH is overexpressed in many tumors of endodermal origin and in at least 95% of CNS tumors compared to normal noncancerous cells. An increase in HAAH gene product in a patient-derived tissue sample (e.g., solid tissue or bodily fluid) is carried out using standard methods, e.g., by Western blot assays or a quantitative assay such as ELISA. For example, a standard competitive ELISA format using an HAAH-specific antibody is used to quantify patient HAAH levels. Alternatively, a sandwich ELISA using a first antibody as the capture antibody and a second HAAH-specific antibody as a detection antibody is used.

Methods of detecting HAAH include contacting a component of a bodily fluid with an HAAH-specific antibody bound to solid matrix, e.g., microtiter plate, bead, dipstick. For example, the solid matrix is dipped into a patient-derived sample of a bodily fluid, washed, and the solid matrix is contacted with a reagent to detect the presence of immune complexes present on the solid matrix.

Proteins in a test sample are immobilized on (e.g., bound to) a solid matrix. Methods and means for covalently or noncovalently binding proteins to solid matrices are known in the art. The nature of the solid surface may vary depending upon the assay format. For assays carried out in microtiter wells, the solid surface is the wall of the microtiter well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates, polyvinylidine fluoride (known as IMMULON™), diazotized paper, nylon membranes, activated beads, and Protein A beads. The solid support containing the antibody is typically washed after contacting it with the test sample, and prior to detection of bound immune complexes. Incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

An HAAH-detection reagent, e.g., an antibody, is packaged in the form of a kit, which contains one or more HAAH-specific antibodies, control formulations (positive and/or negative), and/or a detectable label. The assay may be in the form of a standard two-antibody sandwich assay format known in the art.

Production of HAAH-Specific Antibodies

Anti-HAAH antibodies were obtained by techniques well known in the art. Such antibodies are polyclonal or monoclonal. Polyclonal antibodies were obtained using standard methods, e.g., by the methods described in Ghose et al., Methods in Enzymology, Vol. 93, 326-327, 1983. An HAAH polypeptide, or an antigenic fragment thereof, was used as the immunogen to stimulate the production of polyclonal antibodies in the antisera of rabbits, goats, sheep, or rodents. Antigenic polypeptides for production of both polyclonal and monoclonal antibodies useful as immunogens include polypeptides which contain an HAAH catalytic domain. For example, the immunogenic polypeptide is the full-length mature HAAH protein or an HAAH fragment containing the carboxyterminal catalytic domain e.g., an HAAH polypeptide containing the His motif of SEQ ID NO:2.

Antibodies which bind to the same epitopes as those antibodies disclosed herein are identified using standard methods, e.g., competitive binding assays, known in the art.

Monoclonal antibodies were obtained by standard techniques. Ten μg of purified recombinant HAAH polypeptide was administered to mice intraperitoneally in complete Freund's adjuvant, followed by a single boost intravenously (into the tail vein) 3-5 months after the initial inoculation. Antibody-producing hybridomas were made using standard methods. To identify those hybridomas producing antibodies that were highly specific for an HAAH polypeptide, hybridomas were screened using the same polypeptide immunogen used to immunize. Those antibodies which were identified as having HAAH-binding activity are also screened for the ability to inhibit HAAH catalytic activity using the enzymatic assays described below. Preferably, the antibody has a binding affinity of at least about 10⁸ liters/mole and more preferably, an affinity of at least about 10⁹ liters/mole.

Monoclonal antibodies are humanized by methods known in the art, e.g., MAbs with a desired binding specificity can be commercially humanized (Scotgene, Scotland; Oxford Molecular, Palo Alto, Calif.).

HAAH-specific intrabodies are produced as follows. Following identification of a hybridoma producing a suitable monoclonal antibody, DNA encoding the antibody is cloned. DNA encoding a single chain HAAH-specific antibody in which heavy and light chain variable domains are separated by a flexible linker peptide is cloned into an expression vector using known methods (e.g., Marasco et al., 1993, Proc. Natl. Acad. Sci. USA 90:7889-7893 and Marasco et al., 1997, Gene Therapy 4:11-15). Such constructs are introduced into cells, e.g., using standard gene delivery techniques for intracellular production of the antibodies. Intracellular antibodies, i.e., intrabodies, are used to inhibit signal transduction by HAAH. Intrabodies which bind to a carboxyterminal catalytic domain of HAAH inhibit the ability of HAAH to hydroxylate EGF-like target sequences.

Methods of linking HAAH-specific antibodies (or fragments thereof) which bind to cell surface exposed epitopes of HAAH on the surface of a tumor cell are linked to known cytotoxic agents, e.g., ricin or diptheria toxin, using known methods.

Deposit of Biological Materials

Under the terms of the Budapest Treaty on the International Recognition of the Deposit of Microorganisms for the Purpose of Patent Procedure, hybridoma FB501 (which produces monoclonal antibody FB50), hybridoma HA386A (which produces monoclonal antibody 86A), hybridoma HA15C7A (which produces monoclonal antibody 5C7), and hybridoma HA219B (which produces monoclonal antibody 19B) were deposited on May 17, 2001, with the American Type Culture Collection (ATCC) of 10801 University Boulevard, Manassas, Va. 20110-2209 USA.

Applicants' assignee represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant's assignee acknowledges its duty to replace the deposit should the depository be unable to furnish a sample when requested due to the condition of the deposit.

Methods of Treating Malignant Tumors

Patients with tumors characterized as overexpressing HAAH as such tumors of endodermal origin (e.g., pancreatic tumors) or CNS tumors are preferably treated by administering antibodies which specifically or preferentially bind HAAH polypeptides on the surface of the tumor cell or by administering antisense nucleic acids.

Antisense therapy is used to inhibit expression of HAAH in patients suffering from hepatocellular carcinomas, cholangiocarcinomas, glioblastomas and neuroblastomas. For example, an HAAH antisense strand (either RNA or DNA) is directly introduced into the cells in a form that is capable of binding to the mRNA transcripts. Alternatively, a vector containing a sequence which, which once within the target cells, is transcribed into the appropriate antisense mRNA, may be administered. Antisense nucleic acids which hybridize to target mRNA decrease or inhibit production of the polypeptide product encoded by a gene by associating with the normally single-stranded mRNA transcript, thereby interfering with translation and thus, expression of the protein. For example, DNA containing a promoter, e.g., a tissue-specific or tumor specific promoter, is operably linked to a DNA sequence (an antisense template), which is transcribed into an antisense RNA. By “operably linked” is meant that a coding sequence and a regulatory sequence(s) (i.e., a promoter) are connected in such a way as to permit gene expression when the appropriate molecules (e.g., transcriptional activator proteins) are bound to the regulatory sequence(s).

Oligonucleotides complementary to various portions of HAAH mRNA were tested in vitro for their ability to decrease production of HAAH in tumor cells (e.g., using the FOCUS hepatocellular carcinoma (HCC) cell line) according to standard methods. A reduction in HAAH gene product in cells contacted with the candidate antisense composition compared to cells cultured in the absence of the candidate composition is detected using HAAH-specific antibodies or other detection strategies. Sequences which decrease production of HAAH in in vitro cell-based or cell-free assays are then be tested in vivo in rats or mice to confirm decreased HAAH production in animals with malignant neoplasms.

Antisense therapy is carried out by administering to a patient an antisense nucleic acid by standard vectors and/or gene delivery systems. Suitable gene delivery systems may include liposomes, receptor-mediated delivery systems, naked DNA, and viral vectors such as herpes viruses, retroviruses, adenoviruses and adeno-associated viruses, among others. A reduction in HAAH production results in a decrease in signal transduction via the IRS signal transduction pathway. A therapeutic nucleic acid composition is formulated in a pharmaceutically acceptable carrier. The therapeutic composition may also include a gene delivery system as described above. Pharmaceutically acceptable carriers are biologically compatible vehicles which are suitable for administration to an animal: e.g., physiological saline. A therapeutically effective amount of a compound is an amount which is capable of producing a medically desirable result such as reduced production of an HAAH gene product or a reduction in tumor growth in a treated animal.

Parenteral administration, such as intravenous, subcutaneous, intramuscular, and intraperitoneal delivery routes, may be used to deliver nucleic acids or HAAH-inhibitory peptides or non-peptide compounds. For treatment of CNS tumors, direct infusion into cerebrospinal fluid is useful. The blood-brain barrier may be compromised in cancer patients, allowing systemically administered drugs to pass through the barrier into the CNS. Liposome formulations of therapeutic compounds may also facilitate passage across the blood-brain barrier.

Dosages for any one patient depends upon many factors, including the patient's size, body surface area, age, the particular nucleic acid to be administered, sex, time and route of administration, general health, and other drugs being administered concurrently. Dosage for intravenous administration of nucleic acids is from approximately 106 to 1022 copies of the nucleic acid molecule.

Ribozyme therapy is also be used to inhibit HAAH gene expression in cancer patients. Ribozymes bind to specific mRNA and then cut it at a predetermined cleavage point, thereby destroying the transcript. These RNA molecules are used to inhibit expression of the HAAH gene according to methods known in the art (Sullivan et al., 1994, J. Invest. Derm. 103:85S-89S; Czubayko et al., 1994, J. Biol. Chem. 269:21358-21363; Mahieu et al, 1994, Blood 84:3758-65; Kobayashi et al. 1994, Cancer Res. 54:1271-1275).

HAAH-Specific Antibodies Inhibit Tumor Cell Growth

HAAH-specific antibodies inhibit the proliferation of tumor cells in culture. Two different HAAH-specific antibodies, FB-50 and 5C7, were tested. Tumor cells (a heptatocarcinoma cell line, a lung carcinoma cell line, and a breast carcinoma cell line) were seeded in a 96 well plate and incubated with varying concentrations of antibody for 48 hours. The cells were fixed with acetone. Cell growth was monitored using a sulforhodamine B dye binding assay. The data indicated a reduction in cell viability and proliferation in the presence of FB50 compared to in its absence.

Passive Immunization

The HAAH-specific antibodies described herein are used to inhibit the growth and/or invasiveness of a tumor cell or kill the tumor cell.

Purified antibody preparations (e.g., a purified monoclonal antibody, an antibody fragment, or single chain antibody) is administered to an individual diagnosed with a tumor or at risk of developing a tumor. The antibody preparations are administered using methods known in the art of passive immunization, e.g., intravenously or intramuscularly. The antibodies used in the methods described herein are formulated in a physiologically-acceptable excipient. Such excipients, e.g., physiological saline, are known in the art.

The antibody is preferably a high-affinity antibody, e.g., an IgG-class antibody or fragment or single chain thereof. Alternatively, the antibody is an IgM isotype. Antibodies are monoclonal, e.g., a murine monoclonal antibody or fragment thereof, or a murine monoclonal antibody, which has been humanized. The antibody is a human monoclonal antibody. The affinity of a given monoclonal antibody is further increased using known methods, e.g., by selecting for increasingly higher binding capacity (e.g., according to the method described in Boder et al., 2000, Proc. Natl. Acad. Sci. U.S.A. 97:10701-10705). Optionally, the antibody, antibody fragment, or high affinity single chain antibody is conjugated to a toxic moiety prior to administration. Toxic moieties suitable for conjugation include ricin, Pseudomonas toxin, Diptheria toxin as well as radioisotopes and chemotherapeutic agents known in the art. Such antibody toxins damage or kill a tumor cell upon binding to the tumor cell or upon internalization into the cytoplasm of the tumor cell.

Antibody preparations or antibody-toxin preparations are administered at doses of approximately 0.01-2 mL/kg of body weight. Doses are readministered weekly or monthly as necessary to reduce tumor load in a treated individual.

Active Immunization

Active vaccination is the process of inducing an animal to respond to an antigen. During vaccination, cells, which recognize the antigen (B cells or cytotoxic T cells), are clonally expanded. In addition, the population of helper T cells specific for the antigen also increase. Vaccination also involves specialized antigen presenting cells, which can process the antigen and display it in a form which can stimulate one of the two pathways. Antigen recognition followed by immune cell expansion and activation leads to the production of antigen-specific antibodies and antigen-specific cellular immune responses. Successful immunization is indicated by an increase in the level of HAAH-specific antibody titer in serum of an immunized individual compared to the level prior to immunization. Preferably, the HAAH-specific antibody titer is at least 10%, more preferably at least 50%, more preferably at least 100%, and most preferably 200% greater than the titer prior to immunization.

An individual is immunized with an AAH (e.g., HAAH) polypeptide or a polynucleotide encoding the peptide. For example, a human patient is immunized with full-length 52 kDa HAAH. Standard adjuvant formulations may be simultaneously administered to enhance immunogenicity of the immunizing polypeptide. Alternatively, shorter polypeptides, e.g., immunogenic fragments of HAAH, are used. For example, a polypeptide contains an extracellular catalytic domain of HAAH (e.g., amino acids 650-700 of SEQ ID NO:2). Other immunogenic fragments of HAAH include a fragment contains a binding site for alpha-ketoglutarate, a fragment that lacks a binding site for alpha-ketoglutarate, one which contains a calcium binding site, and one which lacks a binding site for an EGF-like polypeptide.

DNA Vaccine

In addition to standard active vaccination using a peptide antigen, DNA vaccination is used to generate an immune response to HAAH, and in turn to tumor cells, which overexpress HAAH. Although HAAH is overexpressed on malignant cells, an effective immune response is not made by the patient because tumor cells lack appropriate accessory molecules for antigen presentation. The DNA vaccines described herein result in generation of a humoral as well as cellular immunity specific for HAAH (and cells expressing HAAH on their cell surface). For example, not only is an HAAH-specific antibody produced in the immunized individual, HAAH-specific cytotoxic T cells are generated. HAAH-specific cytotoxic T cells kill tumor cells, thereby reducing tumor load in the immunized individual.

A polynucleotide encoding an AAH polypeptide (full-length or an immunogenic fragment of AAH) is introduced into an individual by known methods, e.g., particle bombardment or direct injection via needle. Typically, the antigen (or DNA encoding the antigen) is delivered intramuscularly. The antigen is also directly injected into other tissues, e.g., tumor sites. DNA is taken up by cells at the point of injection. The cell produces proteins, and the proteins stimulate the immune system of the immunized individual resulting, e.g., in generation of an HAAH-specific antibody. Cellular immunity, e.g., cytotoxic T cells, are also generated.

An effective DNA or mRNA dosage is generally be in the range of from about 0.05 micrograms/kg to about 50 mg/kg, usually about 0.005-5 mg/kg of body weight, e.g., 0.5 to 5 mg/kg. The DNA to be administered is naked (in the absence of transfection-facilitating substances) or complexed with compounds, which enhance cellular uptake of the polynucleotide (e.g., charged lipids, lipid complexes or liposomes). For example, the polynucleotide is administered with Lipofectin™ or precipitating agents such as CaPO₄. The transfected cells, e.g., non-proliferating muscle cells, produce the recombinant antigenic polypeptide for at least one month and up to several months, e.g. 3-6 months. Alternatively, transitory expression of a polypeptide is achieved by introducing the polynucleotide construct into a tissue (e.g., non-muscular tissue or tumor tissue). In the latter case, cells of the tissue produce the polypeptide for a shorter period of time, e.g., several days (3-5 days and up to about 20 days). The level of protein or polypeptide expression by target cells is sufficient to induce production of HAAH-specific antibodies. The level of antibody production is measured using standard methods, e.g., evaluation of antibody titer in patient serum, before and after immunization.

The polynucleotides are administered by standard methods, such as by injection into the interstitial space of tissues such as muscles or skin, introduction into the circulation or into body cavities or by inhalation or insufflation. Polynucleotides are injected or otherwise delivered to the animal with a pharmaceutically acceptable liquid carrier, e.g., a liquid carrier, which is aqueous or partly aqueous. The polynucleotides are associated with a liposome (e.g., a cationic or anionic liposome). The polynucleotide includes genetic information necessary for expression by a target cell, such as a promoters.

One advantage of DNA vaccination is that DNA vaccines can result in longer lasting production of the antigenic protein, thereby booster shots reducing or avoiding booster immunizations.

In addition to inducing an immune response, e.g., an HAAH-specific antibody response, by vaccinating with DNA encoding an HAAH polypeptide, a polynucleotide encoding the antibody itself is introduced. An isolated polynucleotide encoding an HAAH-specific antibody, e.g., variable regions of the antibody, is introduced for production of the antibody in situ. The antibody in turn exerts a therapeutic effect at the target site by binding a cell surface antigen, e.g., extracellular HAAH, or by binding to a catalytic domain of HAAH, to inhibit HAAH function.

In Vivo Diagnostic Imaging

The antibodies (antibody fragments, and single chain antibodies) described herein are useful to diagnose the presence of a tumor in tissues as well as bodily fluids. HAAH-specific antibodies are tagged with a detectable label such as a radioisotope or colorimeteric agent. The labeled antibody is administered to an individual at risk of developing cancer or an individual who has previously been diagnosed with cancer. For example, the antibodies are useful to diagnose metastases of a tumor, which has been surgically excised or treated by chemotherapeutic or radiotherapeutic methods. The sensitivity of the method is sufficient to detect micrometastases in tissues such as lymph nodes. Early and sensitive diagnosis of tumors in this manner allows prompt therapeutic intervention.

The labeled antibody is administered to an individual using known methods, e.g., intravenously, or direct injection into solid or soft tissues. The antibody is allowed to distribute throughout the tissue or throughout the body for a period of approximately 1 hour to 72 hours. The whole body of the individual is then imaged using methods known in the art. Alternatively, a small portion of the body, e.g., a tissue site suspected of harboring a tumor, is imaged. An increase in antibody binding, as measured by an increase in detection of the label, over the level of baseline binding (to normal tissue) indicates the presence of a tumor at the site of binding.

Activation of NOTCH Signaling

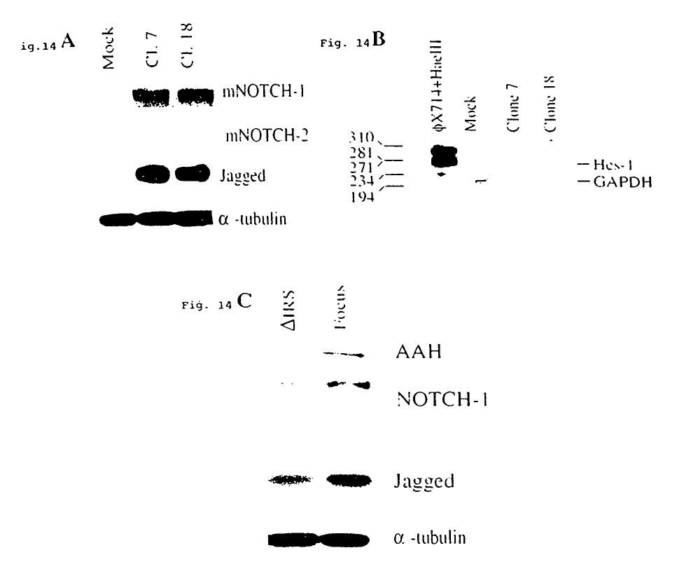

NOTCH signaling is activated in cells highly expressing AAH. FIG. 14A. shows the presence of a 110 kDa NOTCH fragment as revealed by using Western blot. Overexpression of enzymatically active AAH is shown by a display of the 100 kDa cleaved, active NOTCH-1 (Lane 1, Mock DNA transfected clone; Lane 2, clones 7; and Lane 3, clone 18). In contrast, NOTCH-2 was not activated. There was enhanced expression of the full length Jagged ligand in clones expressing AAH as compared to the mock DNA transfected clone. Tubulin was used as internal control for protein loading.

Expression of the Hes-1, a known downstream effector gene, is activated by NOTCH signaling (FIG. 14B). Only AAH-expressing clones activate Notch expression as a transcription factor and subsequently unregulates Hes-1 gene expression as revealed by competitive RT-PCR. Lower panel is an RT-PCR product of GAPDII that served as internal control. FIG. 14C shows expression of human NOTCH-1 (HNOTCH-1) and Jagged-1 where IRS-1 signaling is reduced by a dominant negative mutant (DhIRS-1). Such cells demonstrate downregulation AAH expression and demonstrate a parallel decrease in NOTCH-1 and Jagged levels by Western blot analysis. Tubulin was used as an internal control for protein loading.

Methods of Identifying Compounds that Inhibit HAAH Enzymatic Activity

Aspartyl (asparaginyl) beta-hydroxylaseydroxylase (AAH) activity is measured in vitro or in vivo. For example, HAAH catalyzes posttranslational modification of β carbon of aspartyl and asparaginyl residues of EGF-like polypeptide domains. An assay to identify compounds which inhibit hydroxylase activity is carried out by comparing the level of hydroxylation in an enzymatic reaction in which the candidate compound is present compared to a parallel reaction in the absence of the compound (or a predetermined control value). Standard hydroxylase assays carried out in a test-tube are known in the art, e.g., Lavaissiere et al., 1996, J. Clin. Invest. 98:1313-1323; Jia et al., 1992, J. Biol. Chem. 267:14322-14327; Wang et al., 1991, J. Biol. Chem. 266:14004-14010; or Gronke et al., 1990, J. Biol. Chem. 265:8558-8565. Hydroxylase activity is also measured using carbon dioxide (¹⁴CO₂ capture assay) in a 96-well microtiter plate format (Zhang et al., 1999, Anal. Biochem. 271:137-142. These assays are readily automated and suitable for high throughput screening of candidate compounds to identify those with hydroxylase inhibitory activity.

Candidate compound which inhibit HAAH activation of NOTCH are identified by detecting a reduction in activated NOTCH in a cell which expresses or overexpresses HAAH, e.g., FOCUS HCC cells. The cells are cultured in the presence of a candidate compound. Parallel cultures are incubated in the absence of the candidate compound. To evaluate whether the compound inhibits HAAH activation of NOTCH, translocation of activated NOTCH to the nucleus of the cell is measured. Translocation is measured by detecting a 110 kDa activation fragment of NOTCH in the nucleus of the cell. The activation fragment is cleaved from the large (approximately 300 kDa) transmembrane NOTCH protein upon activation. Methods of measuring NOTCH translocation are known, e.g., those described by Song et al., 1999, Proc. Natl. Acad. Sci U.S.A. 96:6959-6963 or Capobianco et al., 1997, Mol. Cell Biol. 17:6265-6273. A decrease in translocation in the presence of the candidate compound compared to that in the absence of the compound indicates that the compound inhibits HAAH activation of NOTCH, thereby inhibiting NOTCH-mediated signal transduction and proliferation of HAAH-overexpressing tumor cells.

Methods of screening for compounds which inhibit phosphorylation of IRS are carried out by incubating IRS-expressing cells in the presence and absence of a candidate compound and evaluating the level of IRS phosphorylation in the cells. A decrease in phosphorylation in cells cultured in the presence of the compound compared to in the absence of the compound indicates that the compound inhibits IRS-1 phosphorylation, and as a result, growth of HAAH-overexpressing tumors. Alternatively, such compounds are identified in an in vitro phosphorylation assay known in the art, e.g., one which measured phosphorylation of a synthetic substrate such as poly (Glu/Tyr).

### EXAMPLE 1

### Increased Expression of HAAH is Associated with Malignant Transformation

HAAH is a highly conserved enzyme that hydroxylates EGF-like domains in transformation associated proteins. The HAAH gene is overexpressed in many cancer types including human hepatocellular carcinomas and cholangiocarcinomas. HAAH gene expression was found to be undetectable during bile duct proliferation in both human disease and rat models compared to cholangiocarcinoma. Overexpression of HAAH in NIH-3T3 cells was associated with generation of a malignant phenotype, and enzymatic activity was found to be required for cellular transformation. The data described below indicate that overexpression of HAAH is linked to cellular transformation of biliary epithelial cells.

To identify molecules that are specifically overexpressed in transformed malignant cells of human hepatocyte origin, the FOCUS hepatocellular carcinoma (HCC) cell line was used as an immunogen to generate monoclonal antibodies (mAb) that specifically or preferentially recognize proteins associated with the malignant phenotype. A lambda GT11 cDNA expression library derived from HepG2 HCC cells was screened, and a HAAH-specific mAb produced against the FOCUS cell line was found to recognize an epitope on a protein encoded by an HAAH cDNA. The HAAH enzyme was found to be upregulated in several different human transformed cell lines and tumor tissues compared to adjacent human tissue counterparts. The overexpressed HAAH enzyme in different human malignant tissues was found to be catalytically active. HAAH gene expression was examined in proliferating bile ducts and in NIH 3T3 cells. Its role in the generation of the malignant phenotype was measured by the formation of transformed foci, growth in soft agar as an index of anchorage independent growth and tumor formation in nude mice. The role of enzymatic activity in the induction of transformed phenotype was measured by using a cDNA construct with a mutation in the catalytic site that abolished hydroxylase activity. The results indicated that an increase in expression of HAAH gene is associated with malignant transformation of bile ducts.

The following materials and methods were used to generate the data described below.

Antibodies

The FB50 monoclonal antibody was generated by cellular immunization of Balb/C mice with FOCUS HCC cells. A monoclonal anti-Dengue virus antibody was used as a non-relevant control. The HBOH2 monoclonal antibody was generated against a 52 kDa recombinant HAAH polypeptide and recognizes the catalytic domain of beta-hydroxylase from mouse and human proteins. Polyclonal anti-HAAH antibodies cross-react with rat hydroxylase protein. Control antibody anti-Erk-1 was purchased from Santa Cruz Biotechnology, Inc., CA. Sheep anti-mouse and donkey anti-rabbit antisera labeled with horseradish peroxidase were obtained from Amersham, Arlington Heights, Ill.

Constructs

The murine full length AAH construct (pNH376) and the site-directed mutation construct (pNH376-H660) with abolished catalytic activity were cloned into the eukaryotic expression vector pcDNA3 (Invitrogen Corp., San Diego, Calif.). The full length human AAH was cloned into prokaryotic expression vector pBC-SK+ (Stratagene, La Jolla, Calif.). The full length human AAH (GENBANK Accession No. S83325) was subcloned into the EcoRI site of the pcDNA3 vector.

Animal Model of Bile Duct Proliferation

Rats were divided into 9 separate groups of 3 animals each except for group 9, which contained 5 rats. Group 1 was the non-surgical control group, and group 2 was the sham-operated surgical control. The remaining groups underwent common bile duct ligation to induce intrahepatic bile duct proliferation and were evaluated at 6, 12, 24, 48 hours and 4, 8 and 16 days as shown in Table 3. Animals were asphyxiated with CO₂, and liver samples were taken from left lateral and median lobes, fixed in 2% paraformaldehyde and embedded in paraffin. Liver samples (5 μm) were cut and stained with hematoxylin and eosin to evaluate intrahepatic bile duct proliferation. Immunohistochemistry was performed with polyclonal anti-HAAH antibodies that cross-react with the rat protein to determine levels of protein expression.

Bile Duct Proliferation Associated with Primary Sclerosing Cholangitis (PSC)

Liver biopsy samples were obtained from 7 individuals with PSC and associated bile duct proliferation. These individuals were evaluated according to standard gastroenterohepatological protocols. Patients were 22-46 years of age and consisted of 4 males and 3 females. Four had associated inflammatory bowel disease (3 ulcerative colitis and 1 Crohn's colitis). All patients underwent a radiological evaluation including abdominal ultrasonography and endoscopic retrograde cholangiopancreaticography to exclude the diagnosis of extrahepatic biliary obstruction. Tissue sections were prepared from paraffin embedded blocks and were evaluated by hematoxylin and eosin staining for bile duct proliferation. Expression of HAAH was determined by immunohistochemistry using an HAAH-specific monoclonal antibody such as FB50.

Immunohistochemistry

Liver tissue sections (5 μm) were deparaffinized in xylene and rehydrated in graded alcohol. Endogenous peroxidase activity was quenched by a 30-minute treatment with 0.6% H₂O₂ in 60% methanol. Endogenous biotin was masked by incubation with avidin-biotin blocking solutions (Vector Laboratories, Burlingame, Calif.). The FB50 mAb (for PSC samples) and polyclonal anti-HAAH-hydroxylase antibodies (for rat liver samples) were added to slides in a humidified chamber at 4° C. overnight. Immunohistochemical staining was performed using a standard avidin-biotin horseradish peroxidase complex (ABC) method using Vectastain Kits with diaminobenzidine (DAB) as the chromogen according to manufacturer's instructions (Vector Laboratories, Inc., Burlingame, Calif.). Tissue sections were counterstained with hematoxylin, followed by dehydration in ethanol. Sections were examined by a light microscopy for bile duct proliferation and HAAH protein expression. Paraffin sections of cholangiocarcinoma and placenta were used as positive controls, and hepatosteatosis samples were used as a negative controls. To control for antibody binding specificity, adjacent sections were immunostained in the absence of a primary antibody, or using non-relevant antibody to Dengue virus. As a positive control for tissue immunoreactivity, adjacent sections of all specimens were immunostained with monoclonal antibody to glyceraldehyde 3-phosphate dehydrogenase.

Western Blot Analysis

Cell lysates were prepared in a standard radioimmunoprecipitation assay (RIPA) buffer containing protease inhibitors. The total amount of protein in the lysates was determined by Bio-Rad calorimetric assay (Bio Rad, Hercules, Calif.) followed by 10% sodium dodecyl sulphate-polyacrylamide gel electrophoresis (SDS-PAGE), transferred to PVDF membranes, and subjected to Western blot analysis using FB50, HBOH2, anti-Erk-1 (used as an internal control for protein loading) as primary, sheep anti-mouse and donkey anti-rabbit antisera labeled with horseradish peroxidase as secondary antibodies. Antibody binding was detected with enhanced chemiluminescence reagents (SuperSignal, Pierce Chemical Company, Rockford, Ill.) and film autoradiography. The levels of immunoreactivity were measured by volume densitometry using NIH Image software.

Enzymatic Activity Assay

AAH activity was measured in cell lysates using the first EGF-like domain of bovine protein S as substrate where ¹⁴C-labeled alpha-ketogluterate hydroxylates the domain releasing ¹⁴C containing CO₂ according to standard methods, e.g., those described by Jia et al., 1992, J. Biol. Chem. 267:14322-14327; Wang et al., 1991, J. Biol. Chem. 266:14004-14010; or Gronke et al., 1990, J. Biol. Chem. 265:8558-8565. Incubations were carried out at 37° C. for 30 min in a final volume of 40 μl containing 48 μg of crude cell extract protein and 75 μM EGF substrate.

Cell Transfection Studies

The NIH-3T3 cells were cultured in Dulbecco's modified Eagle's medium (DMEM; Mediatech, Washington, D.C.) supplemented with 10% heat-inactivated fetal calf serum (FCS; Sigma Chemical Co., St. Louis, Mo.), 1% L-glutamine, 1% non-essential amino acids and 1% penicillin-streptomycin (GIBCO BRL, Life Technologies, Inc., Grand Island, N.Y.). Subconfluent NIH-3T3 cells (3×10⁵ cells/60-mm dish) were transfected with 10 μg of one of the following plasmids: 1) non-recombinant pcDNA3 vector (Invitrogen Corp., San Diego, Calif.) as a negative control; 2) pNH376-H660, the murine AAH cDNA that was mutated in the catalytic domain and cloned into the pcDNA3 vector driven by a CMV promoter; 3) pNH376, the wild type murine AAH cDNA cloned into the pcDNA3 vector; 4) pCDHH, wild type human AAH cDNA cloned into the pcDNA3 vector; or 5) pLNCX-UPI, a cDNA that encodes v-Src oncogene (positive control). Cells were transfected using the calcium phosphate transfection kit according to manufacturer's instructions (5 Prime-3 Prime, Inc., Boulder, Colo.). Comparison of cellular transfection efficiency was assessed with the various constructs. For this procedure, confluent plates obtained 48 hours after transfection were split and reseeded into 12 separate 6-cm dishes, and 6 of them were made to grow in the presence of 400 μg/ml G-418 (GIBCO BRL, Life Technologies, Inc., Grant Island, N.Y.) containing medium. The number of G-418 resistant foci was determined at 14 days after transfection and used to correct for any variability in transfection efficiency.

Transformation Assay

The NIH-3T3 cells were transfected with the various constructs and allowed to reach confluence after 48 hours as described above. Each 6 cm dish was split and seeded into 12 different 6 cm dishes. While 6 of them were made to grow in the presence of G-418 to detect transfection efficiency, the other six were grown in complete medium without G-418 and with a medium change every 4th day. The number of transformed foci were counted in these plates without G-418 and expressed as transformed foci per μg transfected DNA.

Anchorage-Independent Cell Growth Assay

A limiting dilution technique (0.15 cell/well of a flat bottom 96-well-plate) was performed on transfectants grown in G-418 in order to isolate cell clones with different levels of HAAH activity as measured by Western blot analysis and enzymatic assay of hydroxylase activity. Cloned cell lines (1.0×10⁴ cells) were suspended in complete medium containing 0.4% low-melting agarose (SeaPlaque GTG Agarose; FMC Bioproducts, Rockland, Me.) and laid over a bottom agar mixture consisting of complete medium with 0.53% low-melting agarose. Each clone was assayed in triplicate. The clones were seeded under these conditions and 10 days later the size (positive growth >0.1 mm in diameter) and number of foci were determined.

Tumorigenicity in Nude Mice

The same clones as assessed in the anchorage independent growth assay were injected into nude mice and observed for tumor formation. Tumorigenicity was evaluated using 10 animals in each of 4 groups (Charles River Labs., Wilmington, Mass.). Group 1 received 1×10⁷ cells stably transfected with mock DNA, Group 2-4 received 1×10⁷ cells of clones stable transfected with pNH376 and expressing various levels of murine HAAH protein. Nude mice were kept under pathogen-free conditions in a standard animal facility. Thirty days after tumor cell inoculation, the animals were sacrificed using isofluorane (Aerrane, Anaquest, N.J.) containing chambers and the tumors were carefully removed and weight determined.

Animal Model of Bile Duct Proliferation

Following ligation of the common bile duct, intrahepatic bile duct proliferation was evident at 48 hours. Tissue samples obtained 8 and 16 days following common bile duct ligation revealed extensive bile duct proliferation as shown in Table 3.

TABLE 3
Bile duct proliferation and HAAH expression at different intervals
after common bile duct ligation

| | Surgical | | Immunohisto- |
|---|---|---|---|
| Group | Procedure | Microscopy* | chemistry |
| 1 | no surgery | normal | negative |
| 2 | sham surgery | normal | negative |
| 3 | 6 hours post ligation | normal | negative |
| 4 | 12 hours post ligation | normal | negative |
| 5 | 24 hours post ligation | normal | negative |
| 6 | 48 hours post ligation | minimal bile duct | negative |
| | | prolif. | |
| 7 | 4 days post ligation | moderate bile duct | negative |
| | | prolif. | |
| 8 | 8 days post ligation | extensive bile duct | negative |
| | | prolif. | |
| 9 | 16 days post ligation | extensive bile duct | negative |
| | | prolif. | |

*Investigation was performed under light microscopy following a hematoxylin and eosin staining.

Immunohistochemical staining failed to detect presence of HAAH in proliferating bile ducts at any time. Analysis of HAAH expression in bile ducts derived from sham surgical controls was also negative, while all samples exhibited positive immunoreactivity with control antibodies to glyceraldehyde 3-phosphate dehydrogenase., Thus, bile duct proliferation was not associated with increased HAAH expression in this standard animal model system.

HAAH Expression in PSC

The liver biopsy specimens from patients with PSC exhibited bile duct proliferation accompanied by periductal fibrosis and a mononuclear inflammatory cell infiltrate without evidence of dysplasia. Adjacent sections immunostained with the an HAAH-specific monoclonal antibody had no detectable HAAH immunoreactivity in proliferating bile ducts. In contrast, sections of cholangiocarcinoma that were immunostained simultaneously using the same antibody and detection reagents manifested intense levels of HAAH immunoreactivity in nearly all tumor cells, whereas adjacent sections of the cholangiocarcinomas exhibited a negative immunostaining reaction with monoclonal antibody to Dengue virus. These findings indicate that HAAH expression was associated with malignant transformation rather than non-cancerous cellular proliferation of intrahepatic bile ducts.

HAAH Associated Transformation of NIH-3T3 Cells

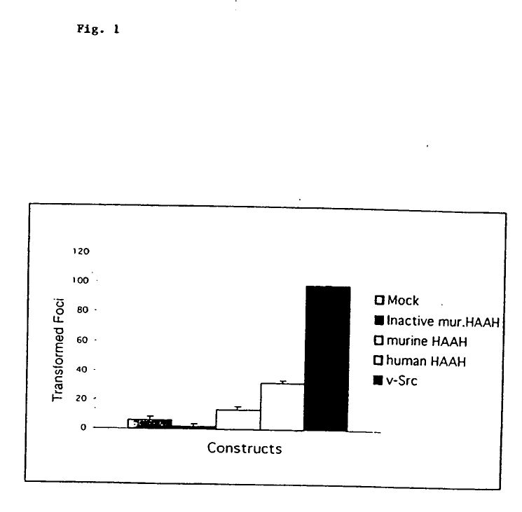

The transforming capability of the murine and human AAH genes, as well as the murine AAH mutant construct without enzymatic activity were compared to mock DNA (negative control) and v-Src transfected NIH-3T3 cells (positive control). The transforming capability of murine AAH was found to be 2-3 times that of vector DNA control as shown in FIG. 1. The transforming capacity of the human gene was greater than that observed with the murine AAH (32±1.5 versus 13±2.6 transformed foci, respectively). The murine and human AAH transfected cells formed large foci, resembling those of v-Src transfected fibroblasts, compared to the occasional much smaller foci observed in cells transfected with vector DNA that displayed the contact inhibition of fibroblast cell lines. Parallel experiments performed using the mutant pNH376-H660 construct without enzymatic activity revealed no transforming activity. This finding indicates that the enzymatic activity of HAAH is required for the transforming activity exhibited by the HAAH gene.

Anchorage-Independent Cell Growth Assay

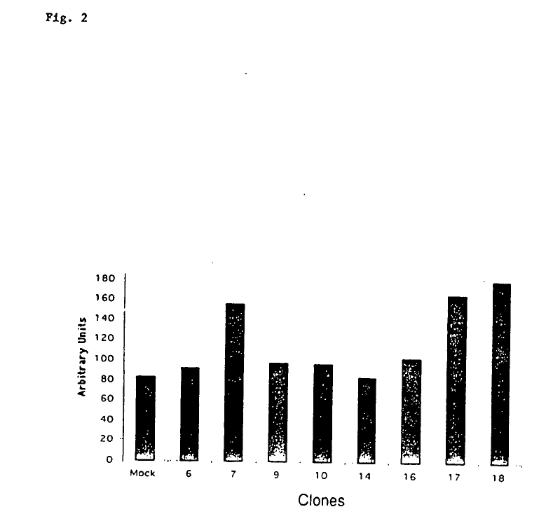

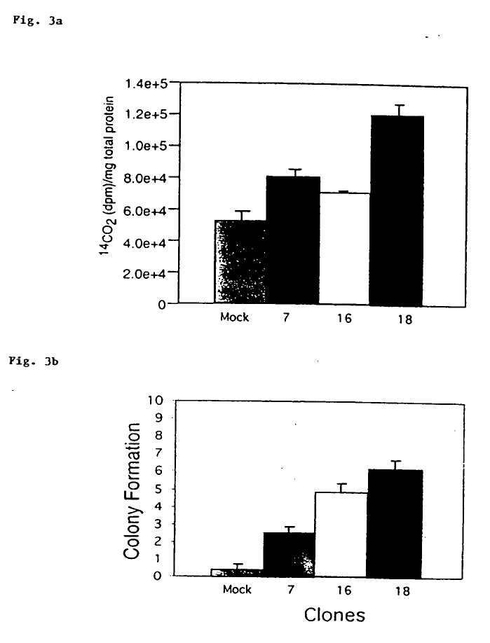

After transient transfection with the murine AAH construct, several different transformed foci were isolated for dilutional cloning experiments to establish stable transfected cell clones with different levels of HAAH gene expression. Nine different cloned cell lines were selected for further study. The expression level of the HAAH protein was determined by Western blot analysis. Clones 7 and 18 had a modest increase in HAAH protein expression, yet formed large colonies in soft agar (FIG. 2). Protein loading was equivalent in all lanes as shown by immunoblotting of the same membranes with an anti-Erk-1 monoclonal antibody. The increased protein expression was associated with increased enzymatic activity as shown in FIG. 3. The capability of these clones to exhibit anchorage independent cell growth in soft agar is presented in FIG. 3. All 3 clones with increased HAAH gene expression demonstrated anchorage independent cell growth compared to the mock DNA transfected clone.

Tumor Formation in Nude Mice

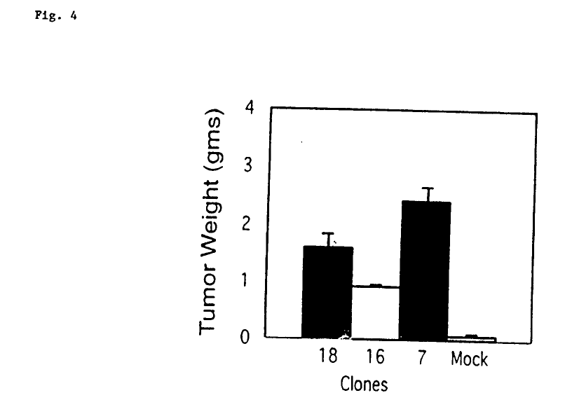

The 3 clones with increased HAAH gene expression were evaluated for the ability to form tumors in nude mice. Tumor size in the mouse given clone 18 was compared to a mock DNA transfected clone. Clones 7, 16 and 18 were highly transformed in this assay and produced large tumors with a mean weight of 2.5, 0.9 and 1.5 grams, respectively (FIG. 4). These data indicate that overexpression of HAAH contributes to induction and maintenance of the malignant phenotype in vivo.

High Level HAAH Expression is Indicative of Malignancy

In order to determine if HAAH expression was associated with malignancy rather than increased cell turnover, two models of bile duct proliferation were studied. In the animal model, ligation of the common bile duct induced extensive intrahepatic bile duct proliferation, yet there was no evidence of HAAH gene expression under these experimental conditions as shown in Table 3. Similarly, HAAH gene expression was assessed in a human disease model associated with bile duct proliferation since PSC is an autoimmune liver disease associated with destruction as well as proliferation of the intra and extrahepatic bile ducts. PSC is premalignant disease, and a significant proportion of affected individuals will eventually develop cholangiocarcinoma. However, no evidence for increased HAAH gene expression in the presence of extensive bile duct proliferation.

Having established that HAAH protein levels were elevated in cholangiocarcinoma and not in normal or proliferating bile ducts, the role of HAAH in the generation of a malignant phenotype was studied. The HAAH gene was transfected into NIH-3T3 cells and cellular changes, e.g., increased formation of transformed foci, colony growth in soft agar and tumor formation in nude mice associated with malignant transformation, were evaluated. The full-length murine and human AAH genes were cloned into expression constructs and transiently transfected into NIH-3T3 cells. An increased number of transformed foci was detected in cells transfected both with the murine and human AAH genes as compared to mock DNA transfected controls. The increased number of transformed foci, after controlling for transfection efficiency, was not as high compared to v-Src gene transfected cells used as a positive control. The enzymatic activity of the HAAH gene was required for a malignant phenotype because a mutant construct which abolished the catalytic site had no transforming properties. Several stable transfectants and cloned NIH-3T3 cell lines with a modest increase in HAAH protein levels and enzymatic activity were established. Such cell lines were placed in soft agar to examine anchorage independent cell growth as another property of the malignant phenotype. All cell lines grew in soft agar compared to mock DNA transfected control, and there was a positive correlation between the cellular level of HAAH gene expression and the number and size of colonies formed. Three of these cloned cell lines formed tumors in nude mice. All three cell lines with increased HAAH expression were oncogenic as shown by the development of large tumors as another well-known characteristic of the transformed phenotype.

To determine whether cellular changes induced by overexpression of HAAH were related to the enzymatic function, a site-directed mutation was introduced into the gene that changed the ferrous iron binding site from histidine to lysine at 660th position of mouse HAAH thereby abolishing hydroxylase activity of the murine HAAH. A corresponding mutation in HAAH is used as a dominant negative mutant to inhibit HAAH hydroxylase activity. The pNH376-H660 construct had no transformation activity indicating cellular changes of the malignant phenotype induced by overexpression depends on the enzymatic activity of the protein.

Notch receptors and their ligands have several EGF-like domains in the N-terminal region that contain the putative consensus sequence for beta-hydroxylation. Notch ligands are important elements of the Notch signal transduction pathway and interaction of Notch with its ligands occurs by means of EGF-like domains of both molecules. Point mutations affecting aspartic acid or asparagine residues in EGF-like domains that are the targets for beta-hydroxylation by HAAH reduce calcium binding and protein-protein interactions involved in the activation of downstream signal transduction pathways. Overexpression of HAAH and Notch protein hydroxylation by HAAH contributes to malignancy. Tumor growth is inhibited by decreasing Notch protein hydroxylation by HAAH.

The data presented herein is evidence that high-level HAAH expression is linked to malignant transformation. An increase in expression of the HAAH cDNA in NIH-3T3 cells induced a transformed phenotype manifested by increased numbers of transformed foci, anchorage-independent growth, and tumorigenesis in nude mice. In addition, intact HAAH-enzyme was found to be required for HAAH-associated transformation. Accordingly, inhibition of as little as 20% of endogenous HAAH enzymatic activity or expression confers a therapeutic benefit. For example, clinical benefit is achieved by 50%-70% inhibition of HAAH expression or activity after administration of an HAAH inhibitory compound compared to the level associated with untreated cancer cell or a normal noncancerous cell.

HAAH is regulated at the level of transcription. Only modest increases in HAAH expression and enzyme activity were required for cellular transformation. These results indicate that increased HAAH gene expression and enzyme activity contribute to the generation or maintenance of the transformed phenotype and that decreasing transcription of the HAAH gene or decreasing enzymatic activity of the HAAH gene product leads to a decrease in malignancy. Accordingly, HAAH transcription is inhibited by administering compounds which decrease binding of Fos and/or Jun (elements which regulate HAAH transcription) to HAAH promoter sequences.

Since HAAH is up-regulated with malignant transformation of bile duct epithelium, and HAAH immunoreactivity is detectable on tumor cell surface membranes, HAAH is also a molecule to which to target a cytotoxic agent, e.g., by linking the cytotoxic agent to a compound that binds to HAAH expressed on the surface of a tumor cell. Assay of HAAH protein levels in either biological fluids such as bile, or cells obtained by fine needle aspiration is a diagnostic marker of human cholangiocarcinoma.

### EXAMPLE 2

### Expression of AAH and Growth and Invasiveness of Malignant CNS Neoplasms

AAH is abundantly expressed in carcinomas and trophoblastic cells, but not in most normal cells, including those of CNS origin. High levels of AAH expression were observed in 15 of 16 glioblastomas, 8 of 9 anaplastic oligodendrogliomas, and 12 of 12 primitive neuroectodermal tumors (PNETs). High levels of AAH immunoreactivity were primarily localized at the infiltrating edges rather than in the central portions of tumors. Double-label immunohistochemical staining demonstrated a reciprocal relationship between AAH and tenascin, a substrate for AAH enzyme activity. PNET2 neuronal cell lines treated with phorbol ester myristate or retinoic acid to stimulate neuritic extension and invasive growth exhibited high levels of AAH expression, whereas H₂O₂-induced neurite retraction resulted in down-regulation of AAH. PNET2 neuronal cells that stably over-expressed the human AAH cDNA had increased levels of PCNA and Bc1-2, and reduced levels of p21/Waf1 and p16, suggesting that AAH overexpression results in enhanced pathological cell proliferation, cell cycle progression, and resistance to apoptosis. In addition, the reduced levels of p16 observed in AAH-transfectants indicate that AAH over-expression confers enhanced invasive growth of neoplastic cells since deletion or down-regulation of the p16 gene correlates with more aggressive and invasive in vivo growth of glioblastomas. Increased AAH immunoreactivity was detected at the infiltrating margins of primary malignant CNS neoplasms, further indicating a role of HAAH in tumor invasiveness.

The following materials and methods were used to generate the data described below.

Analysis of AAH Immunoreactivity in Primary Human Malignant CNS Neoplasms:

AAH immunoreactivity was examined in surgical resection specimens of glioblastoma (N=16), anaplastic oligodendroglioma (N=9), and primitive neuroectodermal tumor (PNET; supratentorial neuroblastomas (N=3) and medulloblastomas (N=9). The histopathological sections were reviewed to confirm the diagnoses using standard criteria. Paraffin sections from blocks that contained representative samples of viable solid tumor, or tumor with adjacent intact tissue were studied. Sections from normal adult postmortem brains (N=4) were included as negative controls. AAH immunoreactivity was detected using qn HAAH-specific monoclonal antibody. Immunoreactivity was revealed by the avidin-biotin horseradish peroxidase complex method (Vector ABC Elite Kit; Vector Laboratories, Burlingame, Calif.) using 3-3′ diaminobenzidine (DAB) as the chromogen (24) and hematoxylin as a counterstain.

Tenascin and laminin are likely substrates for AAH due to the presence of EGF-like repeats within the molecules. Double-immunostaining studies were performed to co-localize AAH with tenascin or laminin. The AAH immunoreactivity was detected by the ABC method with DAB as the chromogen, and tenascin or laminin immunoreactivity was detected by the avidin-biotin alkaline phosphatase complex method (Vector Laboratories, Burlingame, Calif.) with BCIP/NBT as the substrate. As positive and negative controls, adjacent sections were immunostained with monoclonal antibody to glial fibrillary acidic protein (GFAP) and Hepatitis B surface antigen. All specimens were batch immunostained using the same antibody dilutions and immunodetection reagents.

Cell Lines and Culture Conditions

Studies were conducted to determine whether AAH expression was modulated with neurite (filopodia) extension (sprouting) as occurs with invasive growth of malignant neoplasms. Human PNET2 CNS-derived and SH-Sy5y neuroblastoma cells were cultured and stimulated for 0, 1, 2, 3, 5, or 7 days with 100 nM phorbol 12-ester 13-acetate or 10 μM retinoic acid to induce sprouting. In addition, to examine the effects of neurite retraction on AAH expression, subconfluent cultures were treated for 24 hours with low concentrations (10-40 μM) of H₂O₂. For both studies, AAH expression was evaluated by Western blot analysis using the an HAAH-specific antibody.

Generation of PNET2 AAH-Transfected Clones

The full-length human AAH cDNA (SEQ ID NO:3) was ligated into the pcDNA3.1 mammalian expression vector in which gene expression was under the control of a CMV promoter (Invitrogen Corp., San Diego, Calif.). PNET2 cells were transfected with either pHAAH or pcDNA3 (negative control) using Cellfectin reagent (Gibco BRL, Grand Island, N.Y.). Neomycin-resistant clones were selected for study if the constitutive levels of AAH protein expression were increased by at least two-fold relative to control (pcDNA3) as detected by Western blot analysis. To determine how AAH overexpression altered the expression of genes that modulate the transformed phenotype, the levels of proliferating cell nuclear antigen (PCNA), p53, p21/Waf1, Bc1-2, and p16 were measured in cell lysates prepared from subconfluent cultures of AAH (N=5) and pcDNA3 (N=5) stably transfected clones. PCNA was used as marker of cell proliferation. p53, p21/Waf1, and Bc1-2 levels were examined to determine whether cells that over-expressed AAH were more prone to cell cycle progression and more resistant to apoptosis. The levels of p16 were assessed to determine whether AAH over-expression has a role in tumor invasiveness.

Western Blot Analysis

Cells grown in 10 cm² dishes were lysed and homogenized in a standard radioimmunoprecipitation assay RIPA buffer containing protease and phosphatase inhibitors. The supernatants collected after centrifuging the samples at 12,000×g for 10 minutes to remove insoluble debris were used for Western blot analysis. Protein concentration was measured using the BCA assay (Pierce Chemical Co, Rockford, Ill.). Samples containing 60 μg of protein were electrophoresed in sodium dodecyl sulfate polyacrylamide gels (SDS-PAGE) and subjected to Western blot analysis. Replicate blots were probed with the individual antibodies. Immunoreactivity was detected with horseradish peroxidase conjugated IgG (Pierce Chemical Co, Rockford, Ill.) and enhanced chemiluminescence reagents. To quantify the levels of protein expression, non-saturated autoradiographs were subjected to volume densitometry using NIH Image software, version 1.6. Statistical comparisons between pHAAH and pcDNA3 transfected cells were made using Student T tests.

Antibodies

HAAH-specific monoclonal antibody generated against the FOCUS hepatocellular carcinoma cells were used to detect AAH immunoreactivity. Monoclonal antibodies to tenascin, and glial fibrillary acidic protein, and rabbit polyclonal antibody to laminin were purchased from Sigma Co. (St. Louis, Mo.). Rabbit polyclonal antibody to human p16 was purchased from Santa Cruz Biotechnology Inc. (Santa Cruz, Calif.). The 5C3 negative control monoclonal antibody to Hepatitis B surface antigen was generated using recombinant protein and used as a negative control.

AAH Immunoreactivity in Primary Malignant Brains Tumors

AAH immunoreactivity was detected in 15 of 16 glioblastomas, 8 of 9 anaplastic oligodendrogliomas, and all 12 PNETs. AAH immunoreactivity was localized in the cytoplasm, nucleus, and cell processes. The tissue distribution of AAH immunoreactivity was notable for the intense labeling localized at the interfaces between tumor and intact brain, and the conspicuously lower levels of immunoreactivity within the central portions of the tumors. High levels of AAH immunoreactivity were also observed in neoplastic cells distributed in the subpial zones, leptomeninges, Virchow-Robin perivascular spaces, and in individual or small clusters of neoplastic cells that infiltrated the parenchyma. In contrast, AAH immunoreactivity was not detectable in normal brain. The distribution of AAH immunoreactivity appeared not to be strictly correlated with DNA synthesis since the density of nuclei in mitosis (1-5%) was similar in the central and peripheral portions of the tumors.

Relationship Between AAH and Tenascin Immunoreactivity in Glioblastomas

Tenascin is an extracellular matrix-associated antigen expressed in malignant gliomas. Tenascin contains EGF-like domains within the molecule, a substrate for HAAH hydroxylation. To localize AAH in relation to tenascin immunoreactivity in malignant brain tumors, double-label immunohistochemical staining was performed in which AAH was detected using a brown chromogen (DAB), and tenascin, a blue chromogen (BCIP/NBT). Adjacent sections were similarly double-labeled to co-localize AAH with laminin, another EGF domain containing extracellular matrix molecule expressed in the CNS. Intense levels of tenascin immunoreactivity were observed in perivascular connective tissue and in association with glomeruloid proliferation of endothelial cells. The double-labeling studies demonstrated a reciprocal relationship between AAH and tenascin immunoreactivity such that high levels of AAH were associated with low or undetectable tenascin, and low levels of AAH were associated with abundant tenascin immunoreactivity. Although laminins are also likely substrates for AAH enzyme activity due to the EGF repeats within the molecules, double labeling studies revealed only low levels of laminin immunoreactivity throughout the tumors and at interfaces between tumor and intact tissue.

Analysis of AAH Expression in Neuronal Cell Lines Treated with PMA or RA

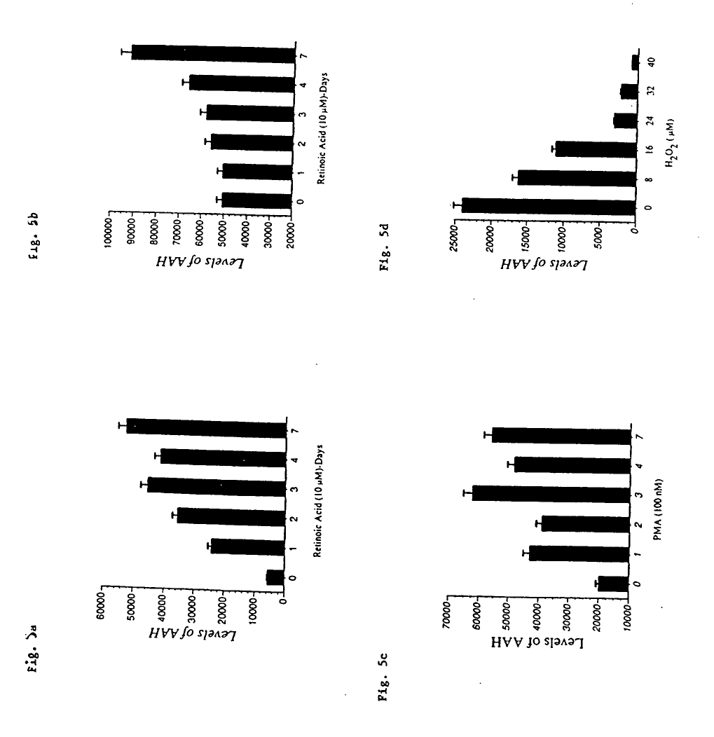

Neuritic sprouting/filopodia extension marks invasive growth of neoplastic neuronal cells. PMA activates protein kinase C signal transduction pathways that are involved in neuritic sprouting. Retinoic acid binds to its own receptor and the ligand-receptor complex translocates to the nucleus where it binds to specific consensus sequences present in the promoter/enhancer regions of target genes involved in neuritic growth. Both PNET2 and SH-Sy5y cells can be induced to sprout by treatment with PMA (60-120 nM) or retinoic acid (5-10 μM). FIGS. 5A-D depict data from representative Western blot autoradiographs; the bar graphs correspond to the mean±S.D. of results obtained from three experiments. Western blot analysis with the FB50 antibody detected doublet bands corresponding to protein with an molecular mass of approximately 85 kDa. Untreated PNET2 cells had relatively low levels of AAH immunoreactivity (FIG. 5A), whereas untreated SH-Sy5y cells had readily detected AAH expression (FIG. 5B). Untreated PNET2 cells exhibited polygonal morphology with coarse, short radial cell processes, whereas SH-SySy cells were slightly elongated and spontaneously extend fine tapered processes. Both cell lines manifested time-dependent increases in the levels of AAH immunoreactivity following either RA (FIGS. 5A and 5B) or PMA (FIG. 5C) stimulation and neurite extension. In PNET2 cells, the levels of AAH protein increased by at least two-fold 24 hours after exposure to RA or PMA, and high levels of AAH were sustained throughout the 7 days of study. In SH-Sy5y cells, the RA- or PMA-stimulated increases in AAH expression occurred more gradually and were highest after 7 days of treatment (FIG. 5B).

To examine the effect of AAH expression on neurite retraction, PNET2 and SH-Sy5y cells were treated with low concentrations (8-40 μM) of H₂O₂. After 24 hours exposure to up to 40 μM H₂O₂, although most cells remained viable (Trypan blue dye exclusion), they exhibited neurite retraction and rounding. Western blot analysis using the FB50 antibody demonstrated H₂O₂ dose-dependent reductions in the levels of AAH protein (FIG. 5D).

Effects of AAH Over-Expression in PNET2 Cells

To directly assess the role of AAH overexpression in relation to the malignant phenotype, PNET2 cells were stably transfected with the human full-length cDNA with gene expression under control of a CMV promoter (pHAAH). Neomycin-resistant clones that had at least two-fold higher levels of AAH immunoreactivity relative to neomycin-resistant pcDNA3 (mock) clones were studied. Since aggressive behavior of malignant neoplasms is associated with increased DNA synthesis, cell cycle progression, resistance to apoptosis, and invasive growth, the changes in phenotype associated with constitutive over-expression of AAH were characterized in relation to PCNA, p21/Waf1, p53, Bc1-2, and p16. PCNA was used as an index of DNA synthesis and cell proliferation. p21/Waf1 is a cell cycle inhibitor. Expression of the p53 tumor-suppressor gene increases prior to apoptosis, whereas bc1-2 inhibits apoptosis and enhances survival of neuronal cells. p16 is an oncosuppressor gene that is often either down-regulated or mutated in infiltrating malignant neoplasms.

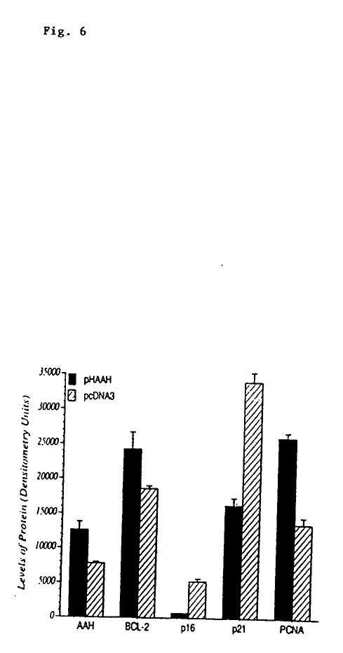

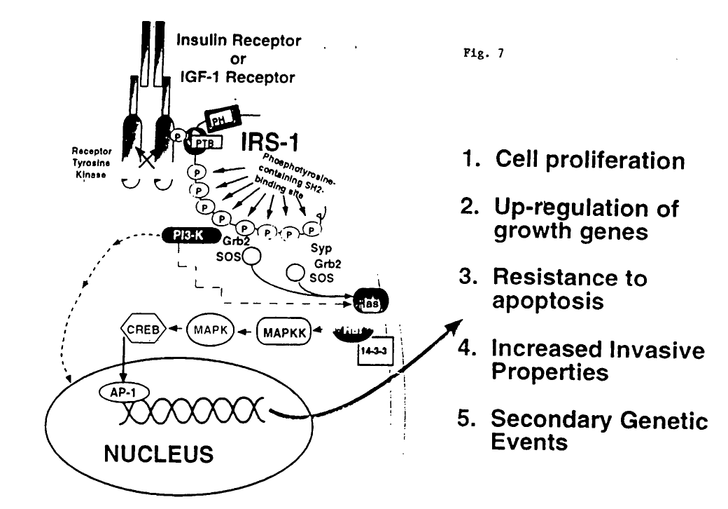

Five pHAAH and 5 pcDNA3 clones were studied. Increased levels of AAH expression in the pHAAH transfected clones was confirmed by Western (FIG. 6) and Northern blot analyses. Western blot analysis using cell lysates from cultures that were 70 to 80 percent confluent demonstrated that constitutively increased levels of AAH expression (approximately 85 kDa; P<0.05) in pHAAH-transfected cells were associated with significantly increased levels of PCNA (approximately 35 kDa; P<0.01) and Bc1-2 (approximately 25 kDa; P<0.05), and reduced levels of p21/Waf1 (approximately 21 kDa; P<0.001) and p16 (approximately 16 kDa; P<0.001) (FIG. 6). However, the pHAAH stable transfectants also exhibited higher levels of wild-type p53 (approximately 53-55 kDa). Although AAH expression (85 kDa protein) in the stable transfectants was increased by only 75 to 100 percent, the levels of p16 and p21/Waf1 were sharply reduced, and PCNA increased by nearly two-fold (FIG. 6).

Increased AAH Expression is Indicative of Growth and Invasiveness of Malignant CNS Neoplasms

The data described herein demonstrates that AAH overexpression is a diagnostic tool by which to identify primary malignant CNS neoplasms of both neuronal and glial cell origin. Immunohistochemical staining studies demonstrated that AAH overexpression was detectable mainly at the interfaces between solid tumor and normal tissue, and in infiltrating neoplastic cells distributed in the subpial zones, leptomeninges, perivascular spaces, and parenchyma. In vitro experiments demonstrated that AAH gene expression was modulated with neurite (filopodium) extension and invasiveness and down-regulated with neurite retraction. In addition, PNET2 cells stably transfected with the AAH cDNA exhibited increased PCNA and bc1-2, and reduced Waf1/p21 and p16 expression. Therefore, AAH overexpression contributes to the transformed phenotype of CNS cells by modulating the expression of other genes that promote cellular proliferation and cell cycle progression, inhibit apoptosis, or enhance tumor cell invasiveness.

The data demonstrated readily detectable AAH mRNA transcripts (4.3 kB and 2.6 kB) and proteins (85 kDa and 50-56 kDa) in PNET2 and SH-Sy5y cells, but not in normal brain. Correspondingly, high levels of AAH immunoreactivity were observed in 35 of the 37 in malignant primary CNS-derived neoplasms studied, whereas the 4 normal control brains had no detectable AAH immunoreactivity. The presence of high-level AAH immunoreactivity at the infiltrating margins and generally not in the central portions of the tumors indicates that AAH overexpression is involved in the invasive growth of CNS neoplasms. Administration of compounds which decrease AAH expression or enzymatic activity inhibits proliferation of CNS tumors which overexpress AAH, as well as metastases of CNS tumors to other tissue types.

The AAH enzyme hydroxylates EGF domains of a number of proteins. Tenascin, an extracellular matrix molecule that is abundantly expressed in malignant gliomas, contains EGF-like domains. Since tenascin promotes tumor cell invasion, its abundant expression in glioblastomas represents an autocrine mechanism of enhanced tumor cell growth vis-a-vis the frequent overexpression of EGF or EGF-like receptors in malignant glial cell neoplasms. Analysis of the functional domains of tenascins indicated that the mitogenic effects of this family of molecules are largely mediated by the fibronectin domains, and that the EGF-like domains inhibit growth, cell process elongation, and matrix invasion. Therefore, hydroxylation of the EGF-like domains by AAH represents an important regulatory factor in tumor cell invasiveness.

Double-label immunohistochemical staining studies demonstrated a reciprocal relationship between AAH and tenascin immunoreactivity such that high levels AAH immunoreactivity present at the margins of tumors were associated with low levels of tenascin, and low levels of AAH were often associated with high levels of tenascin. These observations indicated that AAH hydroxylation of EGF-like domains of tenascin alters the immunoreactivity of tenascin protein, and in so doing, facilitates the invasive growth of malignant CNS neoplasms into adjacent normal tissue and perivascular spaces.

AAH immunoreactivity was examined in PNET2 and SH-Sy5y neuronal cells induced to undergo neurite extension with PMA or retinoic acid, or neurite retraction by exposure to low doses of H₂O₂. AAH expression was sharply increased by PMA- or retinoic acid-induced neurite (filopodium) extension, and inhibited by H₂O₂-induced neurite retraction and cell rounding. Neurite or filopodium extension and attachment to extracellular matrix are required for tumor cell invasion in the CNS. The EGF-like domains of tenascin inhibit neuritic and glial cell growth into the matrix during development.

To directly examine the role of AAH overexpression in relation to the transformed phenotype, genes modulated with DNA synthesis, cell cycle progression, apoptosis, and tumor invasiveness were examined in neuronal cell clones that stably over-expressed the human AAH cDNA. The findings of increased PCNA and reduced Waf1/p21 immunoreactivity indicated that AAH overexpression enhances cellular proliferation and cell cycle progression. In addition, the finding of increased Bc1-2 expression indicated that AAH overexpression contributes to the transformed phenotype by increasing cellular resistance to apoptosis. The apparently contradictory finding of higher levels of p53 in the cells that overexpressed AAH is explained by the observation that high levels of wildtype p53 in immature neuronal cells were associated with neuritic growth (invasiveness) rather than apoptosis. Levels of p16 were reduced (compared to normal cells) or virtually undetectable in cells that constitutively overexpressed AAH; a deletion mutation of the p16 gene has been correlated with invasive growth and more rapid progression of malignant neoplasms, including those of CNS origin. These data indicate that p16 expression is modulated by AAH.

### EXAMPLE 3

### Increased HAAH Production and IRS-Mediated Signal Transduction

IRS-1 mediated signal transduction pathway is activated in 95% of human HCC tumors compared to the adjacent uninvolved liver tissue. HAAH is a downstream effector gene involved in this signal transduction pathway. HAAH gene upregulation is closely associated with overexpression of IRS-1 in HCC tumors as revealed by immunohistochemical staining and Western blot analysis. A high level of HAAH protein is expressed in HCC and cholangiocarcinoma compared to normal hepatocytes and bile ducts. Both of these tumors also exhibit high level expression of IRS-1 by immunohistochemical staining. FOCUS HCC cell clones stably transfected with a C-terminal truncated dominant negative mutant of IRS-1, which blocks insulin and IGF-1 stimulated signal transduction, was associated with a striking reduction in HAAH gene expression in liver. In contrast, transgenic mice overexpressing IRS-1 demonstrate an increase in HAAH gene expression by Western blot analysis. Insulin stimulation of FOCUS HCC cells (20 and 40 U) in serum free medium and after 16 hr of serum starvation demonstrated upregulation of HAAH gene expression. These data indicate that HAAH gene expression is a downstream effector of the IRS-1 signal transduction pathway.

### EXAMPLE 4

### Effects of HAAH Expression Levels on the Characteristics of the Malignant Phenotype

Overexpression of IRS-1 in NIH 3T3 cells induces transformation. The full-length murine HAAH construct was cloned into the pcDNA3 eukaryotic expression vector. A second murine construct encoded HAAH with abolished catalytic activity due to a site directed mutation. The full-length human HAAH cDNA was cloned into the pcDNA3 expression vector as well as a plasmid that encodes v-src which was used as a positive control for transformation activity. Standard methods were used for transfection of NIH 3T3 cells, control for transfection efficiency, assays of HAAH enzymatic activity, transformation by analysis of foci formation, anchorage-independent cell growth assays and analysis of tumorigenicity in nude mice. The data indicated that HAAH overexpression is associated with generation of a malignant phenotype.

TABLE 4
Overexpression of enzymatically active HAAH indicates malignancy

| cDNA | # of foci ± S.D.^{b} | NIH 3T3 clone | # of colonies^{e} |
|---|---|---|---|
| pcDNA3 | 6.0 ± 3.3 | PcDNA | 0.4 ± 0.5 |
| (mock) | | (mock) | |
| murine | 14.0 ± 2.9 | clone 18^{d} | 6.2 ± 2.9 |
| HAAH | | | |
| mutant murine | 1.6 ± 1.0 | clone 16^{e} | 4.7 ± 6.5 |
| HAAH^{a} | | | |
| human | 32.0 ± 5.4 | | |
| HAAH | | | |
| v-scr | 98.0 ± 7.1 | | |

^{a}enzymatically inactive HAAH
^{b}P < 0.01 compared to mock and mutant murine HAAH
^{c}P < 0.001 compared to mock
^{d}Clone 18 is a stable cloned NIH 3T3 cell line that overexpression human HAAH by approximately two fold.
^{e}Clone 16 is a stable cloned NIH 3T3 cell line that overexpresses human HAAH by about 50%.

These data indicate that overexpression of HAAH is associated with formation of transformed foci. Enzymatic activity is required for cellular transformation to occur. Cloned NIH 3T3 cell lines with increased human HAAH gene expression grew as solid tumors in nude mice. HAAH is a downstream effector gene of the IRS-1 signal transduction pathway.

### EXAMPLE 5

### Inhibition of HAAH Gene Expression

The FOCUS HCC cell line from which the human HAAH gene was initially cloned has a level of HAAH expression that is approximately 34 fold higher than that found in normal liver. To make an HAAH antisense construct, the full length human HAAH cDNA was inserted in the opposite orientation into a retroviral vector containing a G418 resistant gene, and antisense RNA was produced in the cells. Shorter HAAH antisense nucleic acids, e.g., those corresponding to exon 1 of the HAAH gene are also used to inhibit HAAH expression.

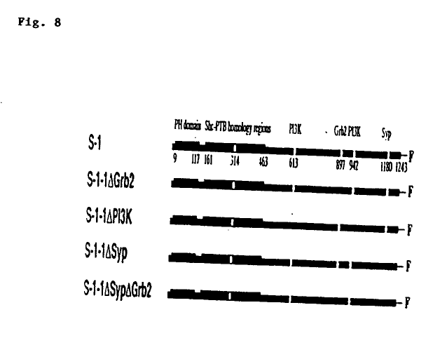

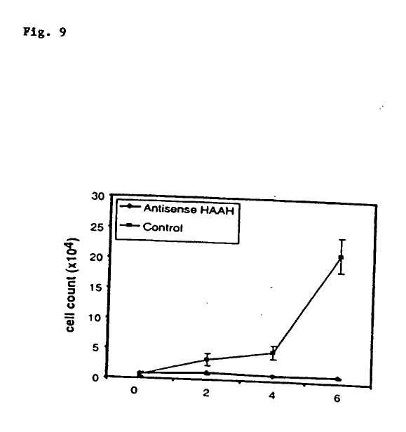

FOCUS cells were infected with this vector and the level of HAAH was determined by Western blot analysis. A reduction in HAAH gene expression was observed. Growth rate and morphologic appearance of cells infected with a retrovirus containing a nonrelevant Green Fluorescent Protein (GFP) also inserted in the opposite orientation as a control (FIG. 8). Cells (harboring the HAAH antisense construct) exhibited a substantial change in morphology characterized by an increase in the cytoplasm to nuclear ratio as well as assuming cell shape changes that were reminiscent of normal adult hepatocytes in culture. Cells with reduced HAAH levels grew at a substantially slower rate than retroviral infected cells expressing antisense (GFP) (control) as shown in FIG. 8. A reduction in HAAH gene expression was associated with a more differentiated noncancerous “hepatocyte like” phenotype. Expression of HAAH antisense sequences are used to inhibit tumor growth rate. Reduction of HAAH cellular levels results in a phenotype characterized by reduced formation of transformed foci, low level or absent anchorage independent growth in soft agar, morphologic features of differentiated hepatocytes as determined by light and phase contrast microscopy, and no tumor formation (as tested by inoculating the cells into nude mice).

### EXAMPLE 6

### Inhibition of AAH Expression by AAH Antisense Oligonucleotides

Oligonucleotides that inhibit AAH gene expression were designed and synthesized using standard methods. For example, antisense oligonucleotides (20 mers) were designed to bind to the 5′ region of the AAH mRNA and overlap with the AUG initiation codon (Table 5). The antisense oligonucleotides were selected such that they were complementary to sequences beginning 1 (Location −1), 6 (Location −6), or 11 (Location −11) nucleotides upstream (prior to) the “A” of the AUG (methionine) codon. In addition, a sense oligonucleotide beginning at Location −3 was made.

TABLE 5
Sequence of exemplary oligonucleotide molecules

| Location (−1) | |
|---|---|
| 5′ CAT TCT TAC GCT GGG CCA TT 3′ | (SEQ ID NO: 10) |
| Location (−6) | |
| 5′ TTA CGC TGG GCC ATT GCA CG 3′ | (SEQ ID NO: 11) |
| Location (−11) | |
| 5′ CTG GGC CAT TGC ACG GTC CG 3′ | (SEQ ID NO: 12) |
| Sense | |
| 5′ ATC ATG CAA TGG CCC AGC GTA A | (SEQ ID NO: 13) |
| 3′ | |

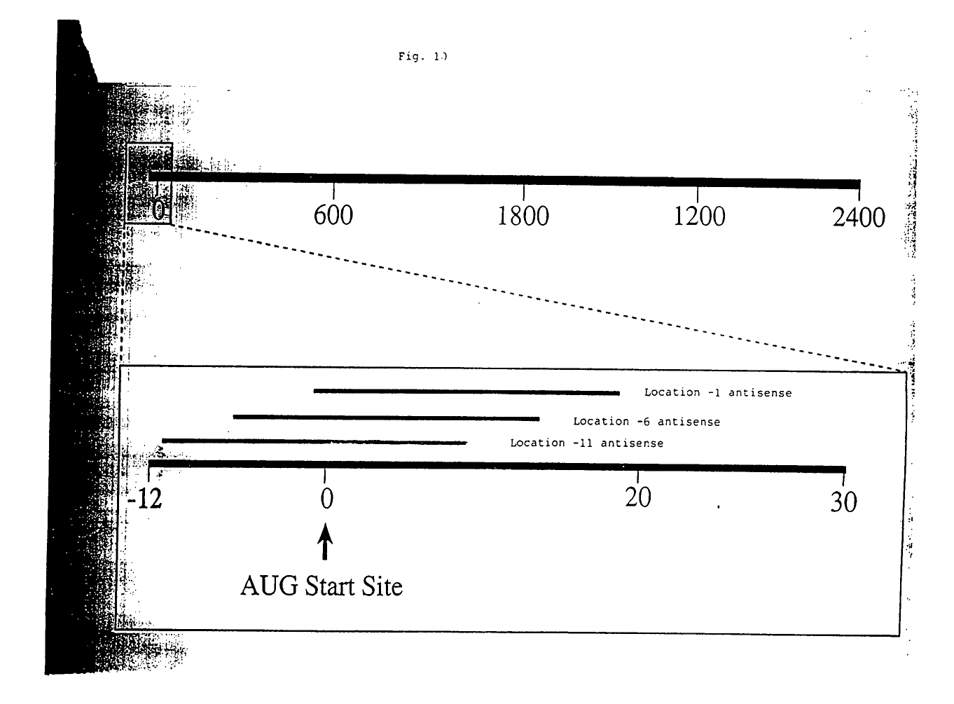

FIG. 10 shows the region of the AAH gene to which the antisense oligonucleotides described in Table 5 bind. All of the oligonucleotides were designed using MacVector 6.5.3 software.

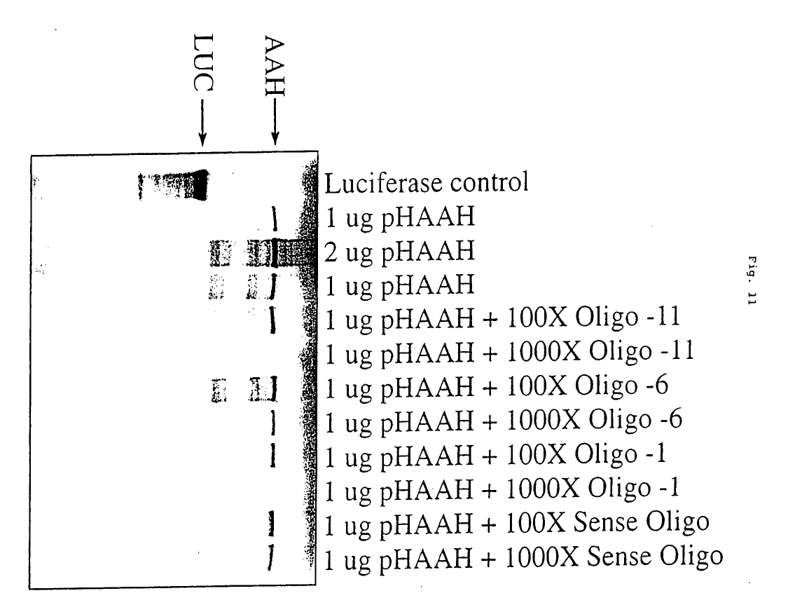

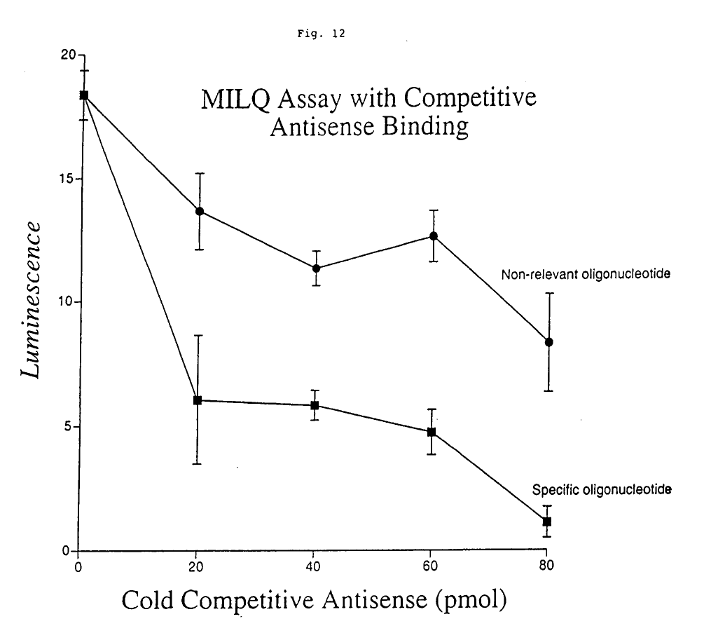

AAH antisense oligonucleotides tested were found to inhibit AAH gene expression. Using an in vitro cell free transcription translation assay (TNT Quick Coupled System), the human AAH cDNA (pHAAH) was used to synthesize AAH protein. In vitro translation was achieved with rabbit reticulocyte lysate included in the reaction mixture. The translated product was labeled with [³⁵S] methionine in the presence of reaction buffer, RNA polymerase, amino acid mixture, and ribonuclease inhibitor (RNAsin). The products were analyzed by SDS-PAGE followed by autoradiography. A luciferase (Luc) expressing plasmid was used as a positive control. In the second and third lanes, synthesis of the ˜85 kD AAH protein is shown (AAH, arrow) using 1 or 2 micrograms of plasmid as the template and the T7 DNA-dependent RNA polymerase primer/promoter to generate mRNA. The addition of 100× or 1000× excess antisense oligonucleotide primer resulted in progressively greater degrees of inhibition of AAH protein synthesis, whereas the inclusion of the same amounts of sense oligonucleotide had no effect on AAH protein synthesis. Further studies demonstrated complete inhibition of AAH protein synthesis only with the antisense oligonucleotides. In addition, effective inhibition of gene expression was observed using all three antisense oligonucleotides tested. FIG. 11 shows the results of an in vitro transcription/translation analysis of AAH antisense oligonucleotides and shows that the antisense oligonucleotides tested block translation of the HAAH RNA and subsequent protein synthesis of HAAH protein.

Inhibition of AAH gene expression was also tested in cells. FIG. 11 shows the results of a Microititer In situ Luminescence Quantification (MILQ) Assay and demonstrates the actual effect of the antisense oligonucleotides inside cells. Substantial reduction in HAAH gene expression was detected by simply adding the antisense oligonucleotides to the culture medium of the cells. The MILQ assay quantifies in situ hybridization binding in cultured cells without the need for RNA extraction. The MILQ assay was used to study competitive antisense binding inhibition to illustrate that the antisense probe hybridized to the mRNA expressed endogenously within the Sh-SySy neuroblastoma cells. In this figure, inhibition of FITC-labeled Location −6 antisense oligonucleotide binding using specific unlabeled antisense oligonucleotides is shown. Minimal inhibition of binding was observed using non-relevant oligonucleotides. The unlabeled specific oligonucleotide was capable of effectively competing for the binding site designated by the FITC-conjugated Location −6 probe, whereas the non-relevant probe exhibited significantly less inhibition at the same molar concentration. Bound probe (FITC-labeled) was detected using horseradish peroxidase conjugated antibodies to FITC, and luminescence reagents were used to detect the bound antibody. Luminescence units were corrected for cell density and are arbitrary in nature. These data indicate that cells effectively take up antisense oligonucleotides in the surrounding environment and that the oligonucleotides taken up effectively and specifically inhibit HAAH gene expression.

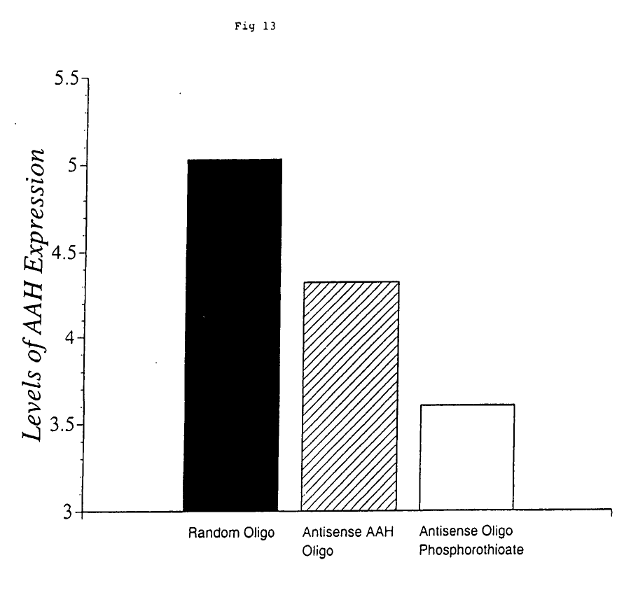

Inhibition of HAAH gene expression is enhanced by contacting cells with a phosphorothioate derivative of the HAAH anti sense. Phosphorothioate anti sense derivatives are made using methods well known in the art. FIG. 13 shows inhibition of AAH gene expression due to antisense (Location −6) oligonucleotide gene delivery into SH-SySy neuroblastoma cells. The MILQ assay was used to measure gene expression resulting from antisense oligonucleotide gene delivery. Cells were contacted with AAH Location −6 antisense DNA, and AAH protein expression was measured using methods known in the art, e.g., the MICE assay (de la Monte, et al, 1999, Biotechniques), to determine if it was inhibited by hybridization with the oligonucleotide. The MICE assay is used to measure immunoreactivity in cultured cells without the need to extract proteins or perform gel electrophoresis. This assay is more sensitive than Western blot analysis. Using the MICE assay, AAH immunoreactivity was assessed in cells transfected with non-relevant (random) oligonucleotide sequences, specific antisense oligonucleotides (Location −6), and a phosphorothioate Location −6 antisense oligonucleotide. Phosphorothioate chemical modification of the oligonucleotide was found to permit greater stability of the DNA inside the cell since the sulfur group protects the DNA from the degradation that normally occurs with. phosphodiester bonds and cellular nucleases. Antisense AAH oligonucleotide (Location −6) transfection resulted in reduced levels of AAH immunoreactivity, and using the phosphorothioate linked Location −6 antisense oligonucleotide, the effect of inhibiting AAH gene expression was substantial relative to the levels observed in cells transfected with the random oligonucleotide. The more effective inhibition of AAH expression using the phosphorothioate-linked antisense oligonucleotide was likely due to the greater stability of the molecule combined, with retained effective binding to mRNA.

### EXAMPLE 7

### Human IRS-1 Mutants

Insulin/IGF-1 stimulated expression of HAAH in HCC cell lines. Dominant-negative IRS-1 cDNAs mutated in the plextrin and phosphotryosine (PTB) domains, and Grb2, Syp and P13K binding motifs located in the C-terminus of the molecule were constructed. Human IRS-1 mutant constructs were generated to evaluate how HAAH gene expression is upregulated by activation of the IRS-1 growth factor signal transduction cascade. Specific mutations in the C terminus of the hIRS-1 molecule abolished the various domains which bind to SH2-effector proteins such as Grb2, Syp and P13K. The human IRS-1 protein contains the same Grb2 and Syp binding motifs of 897YVNI (underlined in Table 5, below and 1180YIDL (underlined in Table 5, below), respectively, as the rat IRS-1 protein. Mutants of hIRS-1 were constructed by substitution of a TAT codon (tyrosine) with a TTT codon (phenylalanine), in these motifs by use of oligonucleotide-directed mutagenesis suing the following primers: (5′-GGGGGAATTTGTCAATA-3′ (SEQ ID NO:8) and 5′-GAATTTGTTAATATTG-3′ (SEQ ID NO:9), respectively). The cDNAs of hIRS-1 (wild-type) and mutants (tyrosine 897-to-phenylalanine and tyrosine 1180-to-phenylalanine) were subcloned into the pBK-CMV expression vector and designated as hIRS-1-wt, 897F, ΔGrb2), 1180F, and ΔSyp.

TABLE 6
Human IRS-1 amino acid sequence

| | |
|---|---|
| MASPPESDGF SDVRKVGYLR KPKSMHKRFF VLRAASEAGG PARLEYYENE KKWRHKSSAP | 61 |
| KRSIPLESCF NINKRADSKN KHLVALYTRD EHFAIAADSE AEQDSWYQAL LQLHNRAKGH | 121 |
| HDGAAALGAG GGGGSCSGSS GLGEAGEDLS YGDVPPGPAF KEVWQVILKP KGLGQTKNLI | 181 |
| GIYRLCLTSK TISFVKLNSE AAAVVLQLMN IRRCGHSENF FFIEVGRSAV TGPGEFWMQV | 241 |
| DDSVVAQNMH ETILEAMRAM SDEFRPRSKS QSSSNCSNPI SVPLRRHHLN NPPPSQVGLT | 301 |
| RRSRTESITA TSPASMVGGK PGSFRVRASS DGEGTMSRPA SVDGSPVSPS TNRTHAHRHR | 361 |
| GSARLHPPLN HSRSIPMPAS RCSPSATSPV SLSSSSTSGH GSTSDCLFPR RSSASVSGSP | 421 |
| SDGGFISSDE YGSSPCDFRS SFRSVTPDSL GHTPPARGEE ELSNYICMGG KGPSTLTAPN | 481 |
| GHYILSRGGN GHRCTPGTGL GTSPALAGDE AASAADLDNR FRKRTHSAGT SPTITHQKTP | 541 |
| SQSSVASIEE YTEMMPAYPP GGGSGGRLPG HRHSAFVPTR SYPEEGLEMH PLERRGGHHR | 601 |
| PDSSTLHTDD GYMPMSPGVA PVPSGRKGSG DYMPMSPKSV SAPQQIINPI RRHPQRVDPN | 661 |
| GYMMMSPSGG CSPDIGGGPS SSSSSSNAVP SGTSYGKLWT NGVGGHHSHV LPHPKPPVES | 721 |
| SGGKLLPCTG DYMNMSPVGD SNTSSPSDCY YGPEDPQHKP VLSYYSLPRS FKHTQRPGEP | 781 |
| EEGARHQHLR LSTSSGRLLY AATADDSSSS TSSDSLGGGY CGARLEPSLP HPHHQVLQPH | 841 |
| LPRKVDTAAQ TNSRLARPTR LSLGDPKAST LPRAREQQQQ QQPLLHPPEP KSPGEYVNIE | 901 |
| FGSDQSGYLS GPVAFHSSPS VRCPSQLQPA PREEETGTEE YMKMDLGPGR RAAWQESTGV | 961 |
| EMGRLGPAPP GAASICRPTR AVPSSRGDYM TMQMSCPRQS YVDTSPAAPV SYADMRTGIA | 1021 |
| AEEVSLPRAT MAAASSSSAA SASPTGPQGA AELAAHSSLL GGPQGPGGMS AFTRVNLSPN | 1081 |
| RNQSAKVIRA DPQGCRRRHS SETFSSTPSA TRVGNTVPFG AGAAVGGGGG SSSSSEDVKR | 1141 |
| HSSASFENVW LRPGELGGAP KEPAKLCGAA GGLENGLNYI DLDLVKDFKQ CPQECTPEPQ | 1201 |
| PPPPPPPHQP LGSGESSSTR RSSEDLSAYA SISFQKQPED RQ | |

(SEQ ID NO: 5; GENBANK Accession No. JS0670; pleckstrin domain spans residues 11-113, inclusive; Phosphate-binding residues include 46, 465, 551, 612, 632, 662, 732, 941, 989, or 1012 of SEQ ID NO: 5)

TABLE 7
Human IRS-1 cDNA

| | |
|---|---|
| cggcggcgcg gtcggagggg gccggcgcgc agagccagac gccgccgctt gttttggttg | 61 |
| gggctctcgg caactctccg aggaggagga ggaggaggga ggaggggaga agtaactgca | 121 |
| gcggcagcgc cctcccgagg aacaggcgtc ttccccgaac ccttcccaaa cctcccccat | 181 |
| cccctctcgc ccttgtcccc tcccctcctc cccagccgcc tggagcgagg ggcagggatg | 241 |
| agtctgtccc tccggccggt ccccagctgc agtggctgcc cggtatcgtt tcgcatggaa | 301 |
| aagccacttt ctccacccgc cgagatgggc ccggatgggg ctgcagagga cgcgcccgcg | 361 |
| ggcggcggca gcagcagcag cagcagcagc agcaacagca acagccgcag cgccgcggtc | 421 |
| tctgcgactg agctggtatt tgggcggctg gtggcggctg ggacggttgg ggggtgggag | 481 |
| gaggcgaagg aggagggaga accccgtgca acgttgggac ttggcaaccc gcctccccct | 541 |
| gcccaaggat atttaatttg cctcgggaat cgctgcttcc agaggggaac tcaggaggga | 601 |
| aggcgcgcgc gcgcgcgcgc tcctggaggg gcaccgcagg gacccccgac tgtcgcctcc | 661 |
| ctgtgccgga ctccagccgg ggcgacgaga gatgcatctt cgctccttcc tggtggcggc | 721 |
| ggcggctgag aggagacttg gctctcggag gatcggggct gccctcaccc cggacgcact | 781 |
| gcctccccgc cggcgtgaag cgcccgaaaa ctccggtcgg gctctctcct gggctcagca | 841 |
| gctgcgtcct ccttcagctg cccctccccg gcgcgggggg cggcgtggat ttcagagtcg | 901 |
| gggtttctgc tgcctccagc cctgtttgca tgtgccgggc cgcggcgagg agcctccgcc | 961 |
| ccccacccgg ttgtttttcg gagcctccct ctgctcagcg ttggtggtgg cggtggcagc | 1021 |
| atggcgagcc ctccggagag cgatggcttc tcggacgtgc gcaaggtggg ctacctgcgc | 1081 |
| aaacccaaga gcatgcacaa acgcttcttc gtactgcgcg cggccagcga ggctgggggc | 1141 |
| ccggcgcgcc tcgagtacta cgagaacgag aagaagtggc ggcacaagtc gagcgccccc | 1201 |
| aaacgctcga tcccccttga gagctgcttc aacatcaaca agcgggctga ctccaagaac | 1261 |
| aagcacctgg tggctctcta cacccgggac gagcactttg ccatcgcggc ggacagcgag | 1321 |
| gccgagcaag acagctggta ccaggctctc ctacagctgc acaaccgtgc taagggccac | 1381 |
| cacgacggag ctgcggccct cggggcggga ggtggtgggg gcagctgcag cggcagctcc | 1441 |
| ggccttggtg aggctgggga ggacttgagc tacggtgacg tgcccccagg acccgcattc | 1501 |
| aaagaggtct ggcaagtgat cctgaagccc aagggcctgg gtcagacaaa gaacctgatt | 1561 |
| ggtatctacc gcctttgcct gaccagcaag accatcagct tcgtgaagct gaactcggag | 1621 |
| gcagcggccg tggtgctgca gctgatgaac atcaggcgct gtggccactc ggaaaacttc | 1681 |
| ttcttcatcg aggtgggccg ttctgccgtg acggggcccg gggagttctg gatgcaggtg | 1741 |
| gatgactctg tggtggccca gaacatgcac gagaccatcc tggaggccat gcgggccatg | 1801 |
| agtgatgagt tccgccctcg cagcaagagc cagtcctcgt ccaactgctc taaccccatc | 1861 |
| agcgtccccc tgcgccggca ccatctcaac aatcccccgc ccagccaggt ggggctgacc | 1921 |
| cgccgatcac gcactgagag catcaccgcc acctccccgg ccagcatggt gggcgggaag | 1981 |
| ccaggctcct tccgtgtccg cgcctccagt gacggcgaag gcaccatgtc ccgcccagcc | 2041 |
| tcggtggacg gcagccctgt gagtcccagc accaacagaa cccacgccca ccggcatcgg | 2101 |
| ggcagcgccc ggctgcaccc cccgctcaac cacagccgct ccatccccat gccggcttcc | 2161 |
| cgctgctcgc cttcggccac cagcccggtc agtctgtcgt ccagtagcac cagtggccat | 2221 |
| ggctccacct cggattgtct cttcccacgg cgatctagtg cttcggtgtc tggttccccc | 2281 |
| agcgatggcg gtttcatctc ctcggatgag tatggctcca gtccctgcga tttccggagt | 2341 |
| tccttccgca gtgtcactcc ggattccctg ggccacaccc caccagcccg cggtgaggag | 2401 |
| gagctaagca actatatctg catgggtggc aaggggccct ccaccctgac cgcccccaac | 2461 |
| ggtcactaca ttttgtctcg gggtggcaat ggccaccgct gcaccccagg aacaggcttg | 2521 |
| ggcacgagtc cagccttggc tggggatgaa gcagccagtg ctgcagatct ggataatcgg | 2581 |
| ttccgaaaga gaactcactc ggcaggcaca tcccctacca ttacccacca gaagaccccg | 2641 |
| tcccagtcct cagtggcttc cattgaggag tacacagaga tgatgcctgc ctacccacca | 2701 |
| ggaggtggca gtggaggccg actgccggga cacaggcact ccgccttcgt gcccacccgc | 2761 |
| tcctacccag aggagggtct ggaaatgcac cccttggagc gtcggggggg gcaccaccgc | 2821 |
| ccagacagct ccaccctcca cacggatgat ggctacatgc ccatgtcccc aggggtggcc | 2881 |
| ccagtgccca gtggccgaaa gggcagtgga gactatatgc ccatgagccc caagagcgta | 2941 |
| tctgccccac agcagatcat caatcccatc agacgccatc cccagagagt ggaccccaat | 3001 |
| ggctacatga tgatgtcccc cagcggtggc tgctctcctg acattggagg tggccccagc | 3061 |
| agcagcagca gcagcagcaa cgccgtccct tccgggacca gctatggaaa gctgtggaca | 3121 |
| aacggggtag ggggccacca ctctcatgtc ttgcctcacc ccaaaccccc agtggagagc | 3181 |
| agcggtggta agctcttacc ttgcacaggt gactacatga acatgtcacc agtgggggac | 3241 |
| tccaacacca gcagcccctc cgactgctac tacggccctg aggaccccca gcacaagcca | 3301 |
| gtcctctcct actactcatt gccaagatcc tttaagcaca cccagcgccc cggggagccg | 3361 |
| gaggagggtg cccggcatca gcacctccgc ctttccacta gctctggtcg ccttctctat | 3421 |
| gctgcaacag cagatgattc ttcctcttcc accagcagcg acagcctggg tgggggatac | 3481 |
| tgcggggcta ggctggagcc cagccttcca catccccacc atcaggttct gcagccccat | 3541 |
| ctgcctcgaa aggtggacac agctgctcag accaatagcc gcctggcccg gcccacgagg | 3601 |
| ctgtccctgg gggatcccaa ggccagcacc ttacctcggg cccgagagca gcagcagcag | 3661 |
| cagcagccct tgctgcaccc tccagagccc aagagcccgg gggaatatgt caatattgaa | 3721 |
| tttgggagtg atcagtctgg ctacttgtct ggcccggtgg ctttccacag ctcaccttct | 3781 |
| gtcaggtgtc catcccagct ccagccagct cccagagagg aagagactgg cactgaggag | 3841 |
| tacatgaaga tggacctggg gccgggccgg agggcagcct ggcaggagag cactggggtc | 3901 |
| gagatgggca gactgggccc tgcacctccc ggggctgcta gcatttgcag gcctacccgg | 3961 |
| gcagtgccca gcagccgggg tgactacatg accatgcaga tgagttgtcc ccgtcagagc | 4021 |
| tacgtggaca cctcgccagc tgcccctgta agctatgctg acatgcgaac aggcattgct | 4081 |
| gcagaggagg tgagcctgcc cagggccacc atggctgctg cctcctcatc ctcagcagcc | 4141 |
| tctgcttccc cgactgggcc tcaaggggca gcagagctgg ctgcccactc gtccctgctg | 4201 |
| gggggcccac aaggacctgg gggcatgagc gccttcaccc gggtgaacct cagtcctaac | 4261 |
| cgcaaccaga gtgccaaagt gatccgtgca gacccacaag ggtgccggcg gaggcatagc | 4321 |
| tccgagactt tctcctcaac acccagtgcc acccgggtgg gcaacacagt gccctttgga | 4381 |
| gcgggggcag cagtaggggg cggtggcggt agcagcagca gcagcgagga tgtgaaacgc | 4441 |
| cacagctctg cttcctttga gaatgtgtgg ctgaggcctg gggagcttgg gggagccccc | 4501 |
| aaggagccag ccaaactgtg tggggctgct gggggtttgg agaatggtct taactacata | 4561 |
| gacctggatt tggtcaagga cttcaaacag tgccctcagg agtgcacccc tgaaccgcag | 4621 |
| cctcccccac ccccaccccc tcatcaaccc ctgggcagcg gtgagagcag ctccacccgc | 4681 |
| cgctcaagtg aggatttaag cgcctatgcc agcatcagtt tccagaagca gccagaggac | 4741 |
| cgtcagtagc tcaactggac atcacagcag aatgaagacc taaatgacct cagcaaatcc | 4801 |
| tcttctaact catgggtacc cagactctaa atatttcatg attcacaact aggacctcat | 4861 |
| atcttcctca tcagtagatg gtacgatgca tccatttcag tttgtttact ttatccaatc | 4921 |
| ctcaggattt cattgactga actgcacgtt ctatattgtg ccaagcgaaa aaaaaaaatg | 4981 |
| cactgtgaca ccagaataat gagtctgcat aaacttcatc ttcaacctta aggacttagc | 5041 |
| tggccacagt gagctgatgt gcccaccacc gtgtcatgag agaatgggtt tactctcaat | 5101 |
| gcattttcaa gatacatttc atctgctgct gaaactgtgt acgacaaagc atcattgtaa | 5161 |
| attatttcat acaaaactgt tcacgttggg tggagagagt attaaatatt taacataggt | 5221 |
| tttgatttat atgtgtaatt ttttaaatga aaatgtaact tttcttacag cacatctttt | 5281 |
| ttttggatgt gggatggagg tatacaatgt tctgttgtaa agagtggagc aaatgcttaa | 5341 |
| aacaaggctt aaaagagtag aatagggtat gatccttgtt ttaagattgt aattcagaaa | 5401 |
| acataatata agaatcatag tgccatagat ggttctcaat tgtatagtta tatttgctga | 5461 |
| tactatctct tgtcatataa acctgatgtt gagctgagtt ccttataaga attaatctta | 5521 |
| attttgtatt ttttcctgta agacaatagg ccatgttaat taaactgaag aaggatatat | 5581 |
| ttggctgggt gttttcaaat gtcagcttaa aattggtaat tgaatggaag caaaattata | 5641 |
| agaagaggaa attaaagtct tccattgcat gtattgtaaa cagaaggaga tgggtgattc | 5701 |
| cttcaattca aaagctctct ttggaatgaa caatgtgggc gtttgtaaat tctggaaatg | 5761 |
| tctttctatt cataataaac tagatactgt tgatctttta aaaaaaaaaa aaaaaaaaaa | 5821 |
| aaaaaaaa | |

(SEQ ID NO: 6; GENBANK Accession No. NM 005544)

The double mutation of tyrosine 897 and 1180 was constructed by replacement of 3′-sequences coding 897F by the same region of 1180F using restriction enzymes NheI and EcoRI, and this construct was called 897F1180F or ΔGrb2 ΔSyp. The expression plasmids were under control of a CMV promoter (hIRS-1-wt, ΔGrb2, ΔSyp, ΔGrb2, ΔSyp and pBK-CMV (mock) and linearized at the 3′-end of poly A signal sequences by MluI restriction enzymes followed by purification. A similar approach was used to change the tyrosine residue to phenyalanine at positions 613 and 942 to create the double P13K mutant construct (ΔPI3K). The hIRS-1 mutants have a FLAG epitope (DYKDDDDK (SEQ ID NO:6)+stop codon) added to the C-terminus by PCR. This strategy allows to distinguish the mutant protein from “wild type” hIRS-1 in stable transfected cell lines. The mutants are used to define the link between the IRS signal transduction pathway and activation of HAAH as a downstream effector gene and identify compounds to inhibit transduction along the pathway to inhibit growth of tumors characterized by HAAH overexpression. Antibodies or other compounds which bind to phosphorylation sites or inhibit phosphorylation at those sites are used to inhibit signal transduction.

### EXAMPLE 8

### HAAH as a Biomarker for Pancreatic Adenocarcinoma Using IHC, qRT-PCR, and ELISA

The identification and establishment of HAAH as a sensitive molecular marker for pancreatic cancer that is detectable early in the course of the disease greatly enhances the early detection of these tumors in high-risk patient populations.

Over-expression of HAAH has been detected by immunohistochemical staining (IHC) in all cancers tested to date (n=18) including lung, liver, colon, pancreas, prostate, ovary, bile duct and breast. HAAH is highly specific for cancer; it has been detected by IHC in>99% of tumor specimens tested (n>1000) and is not present in adjacent non-affected tissue, or in tissue samples from normal individuals. HAAH was evaluated as a biomarker for pancreatic adenocarcinoma utilizing IHC and ELISA as well as qRT-PCR to measure ASPH, the gene that encodes for HAAH. We have developed a sandwich ELISA for the detection of HAAH in serum. Using this assay we have identified HAAH in the serum of patients with breast, ovarian, prostate, colon, esophageal, bladder and kidney cancers. Preliminary results utilizing a 50 ng/mL threshold showed a sensitivity of 94% (n=85) and a specificity of 97% (n=230). In addition, we have developed a quantitative RT-PCR assay to measure the mRNA levels in tumor cells compared to unaffected adjacent and normal cells. Here we have applied these two assays together with IHC to investigate the association of HAAH with pancreatic cancer. Utilizing the serum-based ELISA assay, HAAH was detected in the serum of 18 out of 20 pancreatic cancer samples, 13 having high values and 5 having low values. A panel of ten tissue specimens from patients with a diagnosis of pancreatic cancer along with three samples of adjacent normal tissue were analyzed for HAAH expression by IHC. Two of these adjacent non-cancer specimens displayed striking features of chronic pancreatitis. All 9 pancreatic adenocarcinoma specimens were positive, 1 mucinous cystadenocarcinoma specimen was negative, and the adjacent normal specimen was negative as were the two chronic pancreatitis specimens. Total mRNA was isolated from the same pancreatic specimens used for IHC and the the level of gene expression was determined using the qRT-PCR assay. Detection of ASPH message correlated with a diagnosis of cancer, but not with tumor stage. ASPH message also was higher in moderately differentiated than well-differentiated speciments. Low message levels were found in normal, pancreatitis and mucinous adenocarcinoma specimens, in agreement with the results of the IHC assay. These data show that 1) measurement of HAAH levels has great utility for screening of individuals at risk for pancreatic adenocarcinomas; 2) antibodies against HAAH would likely specifically inhibit the growth and/or invasiveness of cancer cells (e.g., pancreatic cancer cells); and 3) antibodies against HAAH would likely specifically inhibit the activity of HAAH expressed on cancer cells (e.g., pancreatic cancer cells).

U.S. application Ser. No. 09/859,604, filed May 17, 2001 (published as U.S. 20020110559 on Aug. 15, 2002) is incorporated by reference herein in its entirety.

1. A method of conferring an immune response to a tumor cell in a mammal, comprising administering to said mammal an antibody which binds to aspartyl (asparaginyl) beta hydroxylase (HAAH).

2. The method of claim 1, wherein said tumor cell is a pancreatic carcinoma cell.

3. The method of claim 2, wherein said antibody binds to an extracellular domain of HAAH.

4. The method of claim 2, wherein said antibody binds to a catalytic domain of HAAH.

5. The method of claim 2, wherein said catalytic domain comprises amino acids 660-700 of SEQ ID NO:2.

6. The method of claim 2, wherein said antibody is a monoclonal antibody or a fragment thereof.

7. The method of claim 2, wherein said antibody is a high affinity single chain antibody.

8. A method of inhibiting tumor growth in a mammal, comprising administering to said mammal a HAAH-binding antibody conjugated to a cytotoxic agent.

9. The method of claim 8, wherein said cytotoxic agent is onconase or a variant thereof.

10. A method of inducing a HAAH-specific immune response in a mammal, comprising administering to said mammal an HAAH polypeptide.

11. The method of claim 10, wherein said polypeptide comprises the amino acid sequence of SEQ ID NO:2.

12. The method of claim 11, wherein said polypeptide comprises an extracellular domain of HAAH and lacks an intracellular domain of HAAH.

13. The method of claim 11, wherein said polypeptide comprises a catalytic domain of HAAH.

14. The method of claim 11, wherein said polypeptide comprises amino acids 650-700 of SEQ ID NO:2.

15. The method of claim 11, further comprising administering an adjuvant composition.

16. A method for diagnosing a neoplasm in a mammal, comprising contacting a tissue of said mammal with a detectably-labeled antibody which binds to HAAH, wherein an increase in the level of antibody binding at a tissue site compared to the level of binding to a normal normeoplastic tissue indicates the presence of a neoplasm at said tissue site.

17. An isolated antibody or portion thereof that specifically binds to HAAH and inhibits the expression of HAAH on the surface of a pancreatic carcinoma cell.

18. An isolated antibody or portion thereof that specifically binds to HAAH and inhibits the activity of HAAH in a pancreatic carcinoma cell.


SEQUENCE LISTING (13 sequences)

SEQ ID NO: 1 - protein, 36 aa, EGF-like domain consensus sequence
  variant (2)..(8): Wherein Xaa is any amino acid.
  variant (10)..(13): Wherein Xaa is any amino acid.
  variant (15)..(24): Wherein Xaa is any amino acid.
  variant (26)..(26): Wherein Xaa is any amino acid.
  variant (28)..(35): Wherein Xaa is any amino acid.
CXXXXXXXCXXXXCXXXXXXXXXXCXCXXXXXXXXC

SEQ ID NO: 2 - protein, 758 aa, Homo sapiens [sequence omitted]

SEQ ID NO: 3 - DNA, 2324 bp, Homo sapiens
cggaccgtgcaatggcccagcgtaagaatgccaagagcagcggcaacagcagcagcagcggctccggcagcggtagcacgagtgcgggcagcagcagccccggggcccggagagagacaaagcatggaggacacaagaatgggaggaaaggcggactctcgggaacttcattcttcacgtggtttatggtgattgcattgctgggcgtctggacatctgtagctgtcgtttggtttgatcttgttgactatgaggaagttctaggaaaactaggaatctatgatgctgatggtgatggagattttgatgtggatgatgccaaagttttattaggacttaaagagagatctacttcagagccagcagtcccgccagaagaggctgagccacacactgagcccgaggagcaggttcctgtggaggcagaaccccagaatatcgaagatgaagcaaaagaacaaattcagtcccttctccatgaaatggtacacgcagaacatgttgagggagaagacttgcaacaagaagatggacccacaggagaaccacaacaagaggatgatgagtttcttatggcgactgatgtagatgatagatttgagaccctggaacctgaagtatctcatgaagaaaccgagcatagttaccacgtggaagagacagtttcacaagactgtaatcaggatatggaagagatgatgtctgagcaggaaaatccagattccagtgaaccagtagtagaagatgaaagattgcaccatgatacagatgatgtaacataccaagtctatgaggaacaagcagtatatgaacctctagaaaatgaagggatagaaatcacagaagtaactgctccccctgaggataatcctgtagaagattcacaggtaattgtagaagaagtaagcatttttcctgtggaagaacagcaggaagtaccaccagaaacaaatagaaaaacagatgatccagaacaaaaagcaaaagttaagaaaaagaagcctaaacttttaaataaatttgataagactattaaagctgaacttgatgctgcagaaaaactccgtaaaaggggaaaaattgaggaagcagtgaatgcatttaaagaactagtacgcaaataccctcagagtccacgagcaagatatgggaaggcgcagtgtgaggatgatttggctgagaagaggagaagtaatgaggtgctacgtggagccatcgagacctaccaagaggtggccagcctacctgatgtccctgcagacctgctgaagctgagtttgaagcgtcgctcagacaggcaacaatttctaggtcatatgagaggttccctgcttaccctgcagagattagttcaactatttcccaatgatacttccttaaaaaatgaccttggcgtgggatacctcttgataggagataatgacaatgcaaagaaagtttatgaagaggtgctgagtgtgacacctaatgatggctttgctaaagtccattatggcttcatcctgaaggcacagaacaaaattgctgagagcatcccatatttaaaggaaggaatagaatccggagatcctggcactgatgatgggagattttatttccacctgggggatgccatgcagagggttgggaacaaagaggcatataagtggtatgagcttgggcacaagagaggacactttgcatctgtctggcaacgctcactctacaatgtgaatggactgaaagcacagccttggtggaccccaaaagaaacgggctacacagagttagtaaagtctttagaaagaaactggaagttaatccgagatgaaggccttgcagtgatggataaagccaaaggtctcttcctgcctgaggatgaaaacctgagggaaaaaggggactggagccagttcacgctgtggcagcaaggaagaagaaatgaaaatgcctgcaaaggagctcctaaaacctgtaccttactagaaaagttccccgagacaacaggatgcagaagaggacagatcaaatattccatcatgcaccccgggactcacgtgtggccgcacacagggcccacaaactgcaggctccgaatgcacctgggcttggtgattcccaaggaaggctgcaagattcgatgtgccaacgagaccaggacctgggaggaaggcaaggtgctcatctttgatgactcctttgagcacgaggtatggcaggatgcctcatctttccggctgatattcatcgtggatgtgtggcatccggaactgacaccacagcagagacgcagccttccagcaatttagcatgaattcatgcaagcttgggaaactctggagaga

SEQ ID NO: 4 - protein, 31 aa, EGF-like consensus sequence
  variant (3)..(5): Wherein Xaa is any amino acid.
  variant (7)..(8): Wherein Xaa is any amino acid.
  variant (10)..(10): Wherein Xaa is any amino acid.
  variant (14)..(14): Wherein Xaa is any amino acid.
  variant (17)..(18): Wherein Xaa is any amino acid.
  variant (25)..(26): Wherein Xaa is any amino acid.
  variant (29)..(29): Wherein Xaa is any amino acid.
CDXXXCXXKXGNGXCDXXCNNAACXXDGXDC

SEQ ID NO: 5 - protein, 1242 aa, Homo sapiens
MASPPESDGFSDVRKVGYLRKPKSMHKRFFVLRAASEAGGPARLEYYENEKKWRHKSSAPKRSIPLESCFNINKRADSKNKHLVALYTRDEHFAIAADSEAEQDSWYQALLQLHNRAKGHHDGAAALGAGGGGGSCSGSSGLGEAGEDLSYGDVPPGPAFKEVWQVILKPKGLGQTKNLIGIYRLCLTSKTISFVKLNSEAAAVVLQLMNIRRCGHSENFFFIEVGRSAVTGPGEFWMQVDDSVVAQNMHETILEAMRAMSDEFRPRSKSQSSSNCSNPISVPLRRHHLNNPPPSQVGLTRRSRTESITATSPASMVGGKPGSFRVRASSDGEGTMSRPASVDGSPVSPSTNRTHAHRHRGSARLHPPLNHSRSIPMPASRCSPSATSPVSLSSSSTSGHGSTSDCLFPRRSSASVSGSPSDGGFISSDEYGSSPCDFRSSFRSVTPDSLGHTPPARGEEELSNYICMGGKGPSTLTAPNGHYILSRGGNGHRCTPGTGLGTSPALAGDEAASAADLDNRFRKRTHSAGTSPTITHQKTPSQSSVASIEEYTEMMPAYPPGGGSGGRLPGHRHSAFVPTRSYPEEGLEMHPLERRGGHHRPDSSTLHTDDGYMPMSPGVAPVPSGRKGSGDYMPMSPKSVSAPQQIINPIRRHPQRVDPNGYMMMSPSGGCSPDIGGGPSSSSSSSNAVPSGTSYGKLWTNGVGGHHSHVLPHPKPPVESSGGKLLPCTGDYMNMSPVGDSNTSSPSDCYYGPEDPQHKPVLSYYSLPRSFKHTQRPGEPEEGARHQHLRLSTSSGRLLYAATADDSSSSTSSDSLGGGYCGARLEPSLPHPHHQVLQPHLPRKVDTAAQTNSRLARPTRLSLGDPKASTLPRAREQQQQQQPLLHPPEPKSPGEYVNIEFGSDQSGYLSGPVAFHSSPSVRCPSQLQPAPREEETGTEEYMKMDLGPGRRAAWQESTGVEMGRLGPAPPGAASICRPTRAVPSSRGDYMTMQMSCPRQSYVDTSPAAPVSYADMRTGIAAEEVSLPRATMAAASSSSAASASPTGPQGAAELAAHSSLLGGPQGPGGMSAFTRVNLSPNRNQSAKVIRADPQGCRRRHSSETFSSTPSATRVGNTVPFGAGAAVGGGGGSSSSSEDVKRHSSASFENVWLRPGELGGAPKEPAKLCGAAGGLENGLNYIDLDLVKDFKQCPQECTPEPQPPPPPPPHQPLGSGESSSTRRSSEDLSAYASISFQKQPEDRQ

SEQ ID NO: 6 - DNA, 5828 bp, Homo sapiens
cggcggcgcggtcggagggggccggcgcgcagagccagacgccgccgcttgttttggttggggctctcggcaactctccgaggaggaggaggaggagggaggaggggagaagtaactgcagcggcagcgccctcccgaggaacaggcgtcttccccgaacccttcccaaacctcccccatcccctctcgcccttgtcccctcccctcctccccagccgcctggagcgaggggcagggatgagtctgtccctccggccggtccccagctgcagtggctgcccggtatcgtttcgcatggaaaagccactttctccacccgccgagatgggcccggatggggctgcagaggacgcgcccgcgggcggcggcagcagcagcagcagcagcagcagcaacagcaacagccgcagcgccgcggtctctgcgactgagctggtatttgggcggctggtggcggctgggacggttggggggtgggaggaggcgaaggaggagggagaaccccgtgcaacgttgggacttggcaacccgcctccccctgcccaaggatatttaatttgcctcgggaatcgctgcttccagaggggaactcaggagggaaggcgcgcgcgcgcgcgcgctcctggaggggcaccgcagggacccccgactgtcgcctccctgtgccggactccagccggggcgacgagagatgcatcttcgctccttcctggtggcggcggcggctgagaggagacttggctctcggaggatcggggctgccctcaccccggacgcactgcctccccgccggcgtgaagcgcccgaaaactccggtcgggctctctcctgggctcagcagctgcgtcctccttcagctgcccctccccggcgcggggggcggcgtggatttcagagtcggggtttctgctgcctccagccctgtttgcatgtgccgggccgcggcgaggagcctccgccccccacccggttgtttttcggagcctccctctgctcagcgttggtggtggcggtggcagcatggcgagccctccggagagcgatggcttctcggacgtgcgcaaggtgggctacctgcgcaaacccaagagcatgcacaaacgcttcttcgtactgcgcgcggccagcgaggctgggggcccggcgcgcctcgagtactacgagaacgagaagaagtggcggcacaagtcgagcgcccccaaacgctcgatcccccttgagagctgcttcaacatcaacaagcgggctgactccaagaacaagcacctggtggctctctacacccgggacgagcactttgccatcgcggcggacagcgaggccgagcaagacagctggtaccaggctctcctacagctgcacaaccgtgctaagggccaccacgacggagctgcggccctcggggcgggaggtggtgggggcagctgcagcggcagctccggccttggtgaggctggggaggacttgagctacggtgacgtgcccccaggacccgcattcaaagaggtctggcaagtgatcctgaagcccaagggcctgggtcagacaaagaacctgattggtatctaccgcctttgcctgaccagcaagaccatcagcttcgtgaagctgaactcggaggcagcggccgtggtgctgcagctgatgaacatcaggcgctgtggccactcggaaaacttcttcttcatcgaggtgggccgttctgccgtgacggggcccggggagttctggatgcaggtggatgactctgtggtggcccagaacatgcacgagaccatcctggaggccatgcgggccatgagtgatgagttccgccctcgcagcaagagccagtcctcgtccaactgctctaaccccatcagcgtccccctgcgccggcaccatctcaacaatcccccgcccagccaggtggggctgacccgccgatcacgcactgagagcatcaccgccacctccccggccagcatggtgggcgggaagccaggctccttccgtgtccgcgcctccagtgacggcgaaggcaccatgtcccgcccagcctcggtggacggcagccctgtgagtcccagcaccaacagaacccacgcccaccggcatcggggcagcgcccggctgcaccccccgctcaaccacagccgctccatccccatgccggcttcccgctgctcgccttcggccaccagcccggtcagtctgtcgtccagtagcaccagtggccatggctccacctcggattgtctcttcccacggcgatctagtgcttcggtgtctggttcccccagcgatggcggtttcatctcctcggatgagtatggctccagtccctgcgatttccggagttccttccgcagtgtcactccggattccctgggccacaccccaccagcccgcggtgaggaggagctaagcaactatatctgcatgggtggcaaggggccctccaccctgaccgcccccaacggtcactacattttgtctcggggtggcaatggccaccgctgcaccccaggaacaggcttgggcacgagtccagccttggctggggatgaagcagccagtgctgcagatctggataatcggttccgaaagagaactcactcggcaggcacatcccctaccattacccaccagaagaccccgtcccagtcctcagtggcttccattgaggagtacacagagatgatgcctgcctacccaccaggaggtggcagtggaggccgactgccgggacacaggcactccgccttcgtgcccacccgctcctacccagaggagggtctggaaatgcaccccttggagcgtcggggggggcaccaccgcccagacagctccaccctccacacggatgatggctacatgcccatgtccccaggggtggccccagtgcccagtggccgaaagggcagtggagactatatgcccatgagccccaagagcgtatctgccccacagcagatcatcaatcccatcagacgccatccccagagagtggaccccaatggctacatgatgatgtcccccagcggtggctgctctcctgacattggaggtggccccagcagcagcagcagcagcagcaacgccgtcccttccgggaccagctatggaaagctgtggacaaacggggtagggggccaccactctcatgtcttgcctcaccccaaacccccagtggagagcagcggtggtaagctcttaccttgcacaggtgactacatgaacatgtcaccagtgggggactccaacaccagcagcccctccgactgctactacggccctgaggacccccagcacaagccagtcctctcctactactcattgccaagatcctttaagcacacccagcgccccggggagccggaggagggtgcccggcatcagcacctccgcctttccactagctctggtcgccttctctatgctgcaacagcagatgattcttcctcttccaccagcagcgacagcctgggtgggggatactgcggggctaggctggagcccagccttccacatccccaccatcaggttctgcagccccatctgcctcgaaaggtggacacagctgctcagaccaatagccgcctggcccggcccacgaggctgtccctgggggatcccaaggccagcaccttacctcgggcccgagagcagcagcagcagcagcagcccttgctgcaccctccagagcccaagagcccgggggaatatgtcaatattgaatttgggagtgatcagtctggctacttgtctggcccggtggctttccacagctcaccttctgtcaggtgtccatcccagctccagccagctcccagagaggaagagactggcactgaggagtacatgaagatggacctggggccgggccggagggcagcctggcaggagagcactggggtcgagatgggcagactgggccctgcacctcccggggctgctagcatttgcaggcctacccgggcagtgcccagcagccggggtgactacatgaccatgcagatgagttgtccccgtcagagctacgtggacacctcgccagctgcccctgtaagctatgctgacatgcgaacaggcattgctgcagaggaggtgagcctgcccagggccaccatggctgctgcctcctcatcctcagcagcctctgcttccccgactgggcctcaaggggcagcagagctggctgcccactcgtccctgctggggggcccacaaggacctgggggcatgagcgccttcacccgggtgaacctcagtcctaaccgcaaccagagtgccaaagtgatccgtgcagacccacaagggtgccggcggaggcatagctccgagactttctcctcaacacccagtgccacccgggtgggcaacacagtgccctttggagcgggggcagcagtagggggcggtggcggtagcagcagcagcagcgaggatgtgaaacgccacagctctgcttcctttgagaatgtgtggctgaggcctggggagcttgggggagcccccaaggagccagccaaactgtgtggggctgctgggggtttggagaatggtcttaactacatagacctggatttggtcaaggacttcaaacagtgccctcaggagtgcacccctgaaccgcagcctcccccacccccaccccctcatcaacccctgggcagcggtgagagcagctccacccgccgctcaagtgaggatttaagcgcctatgccagcatcagtttccagaagcagccagaggaccgtcagtagctcaactggacatcacagcagaatgaagacctaaatgacctcagcaaatcctcttctaactcatgggtacccagactctaaatatttcatgattcacaactaggacctcatatcttcctcatcagtagatggtacgatgcatccatttcagtttgtttactttatccaatcctcaggatttcattgactgaactgcacgttctatattgtgccaagcgaaaaaaaaaaatgcactgtgacaccagaataatgagtctgcataaacttcatcttcaaccttaaggacttagctggccacagtgagctgatgtgcccaccaccgtgtcatgagagaatgggtttactctcaatgcattttcaagatacatttcatctgctgctgaaactgtgtacgacaaagcatcattgtaaattatttcatacaaaactgttcacgttgggtggagagagtattaaatatttaacataggttttgatttatatgtgtaattttttaaatgaaaatgtaacttttcttacagcacatcttttttttggatgtgggatggaggtatacaatgttctgttgtaaagagtggagcaaatgcttaaaacaaggcttaaaagagtagaatagggtatgatccttgttttaagattgtaattcagaaaacataatataagaatcatagtgccatagatggttctcaattgtatagttatatttgctgatactatctcttgtcatataaacctgatgttgagctgagttccttataagaattaatcttaattttgtattttttcctgtaagacaataggccatgttaattaaactgaagaaggatatatttggctgggtgttttcaaatgtcagcttaaaattggtaattgaatggaagcaaaattataagaagaggaaattaaagtcttccattgcatgtattgtaaacagaaggagatgggtgattccttcaattcaaaagctctctttggaatgaacaatgtgggcgtttgtaaattctggaaatgtctttctattcataataaactagatactgttgatcttttaaaaaaaaaaaaaaaaaaaaaaaaaaaaa

SEQ ID NO: 7 - protein, 8 aa, FLAG epitope
DYKDDDDK

SEQ ID NO: 8 - DNA, 17 bp, hIRS-1 mutagenesis primer
gggggaatttgtcaata

SEQ ID NO: 9 - DNA, 16 bp, hIRS-1 mutagenesis primer
gaatttgttaatattg

SEQ ID NO: 10 - DNA, 20 bp, Exemplary oligonucleotide that inhibits AAH gene expression
cattcttacgctgggccatt

SEQ ID NO: 11 - DNA, 20 bp, Exemplary oligonucleotide that inhibits AAH gene expression
ttacgctgggccattgcacg

SEQ ID NO: 12 - DNA, 20 bp, Exemplary oligonucleotode that inhibits AAH gene expression
ctgggccattgcacggtccg

SEQ ID NO: 13 - DNA, 22 bp, Exemplary oligonucleotide that inhibits AAH gene expression.
atcatgcaatggcccagcgtaa